SEQUENCE LISTING (28 sequences)

SEQ ID NO: 1 - DNA, 20 bp, synthetic construct
  misc_feature 1..20: note = AP2alpha Sense Primer
  source 1..20: mol_type = other DNA
atgctttggaaattgacgga

SEQ ID NO: 2 - DNA, 20 bp, synthetic construct
  misc_feature 1..20: note = AP2alpha Anti-Sense Primer
  source 1..20: mol_type = other DNA
attgacctacagtgcccagc

SEQ ID NO: 3 - DNA, 20 bp, synthetic construct
  misc_feature 1..20: note = Edn3 Sense Primer
  source 1..20: mol_type = other DNA
gcacgtgcttcacctacaag

SEQ ID NO: 4 - DNA, 19 bp, synthetic construct
  misc_feature 1..19: note = Edn3 Anti-Sense Primer
  source 1..19: mol_type = other DNA
ggacagtccatagggcacc

SEQ ID NO: 5 - DNA, 21 bp, synthetic construct
  misc_feature 1..21: note = GAPDH Sense Primer
  source 1..21: mol_type = other DNA
cccatcaccatcttccaggag

SEQ ID NO: 6 - DNA, 22 bp, synthetic construct
  misc_feature 1..22: note = GAPDH Anti-Sense Primer
  source 1..22: mol_type = other DNA
cttctccatggtggtgaagacg

SEQ ID NO: 7 - DNA, 21 bp, synthetic construct
  misc_feature 1..21: note = GDNF Sense Primer
  source 1..21: mol_type = other DNA
tccatgacatcatcgaactga

SEQ ID NO: 8 - DNA, 18 bp, synthetic construct
  misc_feature 1..18: note = GDNF Anti-Sense Primer
  source 1..18: mol_type = other DNA
gtctgcctggtgctgctc

SEQ ID NO: 9 - DNA, 20 bp, synthetic construct
  misc_feature 1..20: note = HoxA2 Sense Primer
  source 1..20: mol_type = other DNA
ccaagaaaaccgcacttctg

SEQ ID NO: 10 - DNA, 17 bp, synthetic construct
  misc_feature 1..17: note = HoxA2 Anti-Sense Primer
  source 1..17: mol_type = other DNA
catcggcgatttccagg

SEQ ID NO: 11 - DNA, 19 bp, synthetic construct
  misc_feature 1..19: note = HoxB3 Sense Primer
  source 1..19: mol_type = other DNA
cgtcatgaatgggatctgc

SEQ ID NO: 12 - DNA, 20 bp, synthetic construct
  misc_feature 1..20: note = HoxB3 Anti-Sense Primer
  source 1..20: mol_type = other DNA
atattcacatcgagccccag

SEQ ID NO: 13 - DNA, 20 bp, synthetic construct
  misc_feature 1..20: note = HoxB5 Sense Primer
  source 1..20: mol_type = other DNA
ggaagcttcacatcagccat

SEQ ID NO: 14 - DNA, 20 bp, synthetic construct
  misc_feature 1..20: note = HoxB5 Anti-Sense Primer
  source 1..20: mol_type = other DNA
ggaactccttttccagctcc

SEQ ID NO: 15 - DNA, 20 bp, synthetic construct
  misc_feature 1..20: note = HoxB7 Sense Primer
  source 1..20: mol_type = other DNA
aacttccggatctacccctg

SEQ ID NO: 16 - DNA, 20 bp, synthetic construct
  misc_feature 1..20: note = HoxB7 Anti-Sense Primer
  source 1..20: mol_type = other DNA
ctttctccagctccagggtc

SEQ ID NO: 17 - DNA, 20 bp, synthetic construct
  misc_feature 1..20: note = Pax3 Sense Primer
  source 1..20: mol_type = other DNA
gccgcatcctgagaagtaaa

SEQ ID NO: 18 - DNA, 20 bp, synthetic construct
  misc_feature 1..20: note = Pax3 Anti-Sense Primer
  source 1..20: mol_type = other DNA
cttcatctgattggggtgct

SEQ ID NO: 19 - DNA, 18 bp, synthetic construct
  misc_feature 1..18: note = Pax7 Sense Primer
  source 1..18: mol_type = other DNA
caaacacagcatcgacgg

SEQ ID NO: 20 - DNA, 20 bp, synthetic construct
  misc_feature 1..20: note = Pax7 Anti-Sense Primer
  source 1..20: mol_type = other DNA
cttcagtgggaggtcaggtt

SEQ ID NO: 21 - DNA, 20 bp, synthetic construct
  misc_feature 1..20: note = Snail2 Sense Primer
  source 1..20: mol_type = other DNA
tgacctgtctgcaaatgctc

SEQ ID NO: 22 - DNA, 19 bp, synthetic construct
  misc_feature 1..19: note = Snail2 Anti-Sense Primer
  source 1..19: mol_type = other DNA
cagaccctggttgcttcaa

SEQ ID NO: 23 - DNA, 18 bp, synthetic construct
  misc_feature 1..18: note = Sox10 Sense Primer
  source 1..18: mol_type = other DNA
agctcagcaagacgctgg

SEQ ID NO: 24 - DNA, 20 bp, synthetic construct
  misc_feature 1..20: note = Sox10 Anti-Sense Primer
  source 1..20: mol_type = other DNA
ctttcttgtgctgcatacgg

SEQ ID NO: 25 - DNA, 20 bp, synthetic construct
  misc_feature 1..20: note = Sox9 Sense Primer
  source 1..20: mol_type = other DNA
gtaatccgggtggtccttct

SEQ ID NO: 26 - DNA, 19 bp, synthetic construct
  misc_feature 1..19: note = Sox9 Anti-Sense Primer
  source 1..19: mol_type = other DNA
gtacccgcacttgcacaac

SEQ ID NO: 27 - DNA, 20 bp, synthetic construct
  misc_feature 1..20: note = Zic1 Sense Primer
  source 1..20: mol_type = other DNA
aagatccacaaaaggacgca

SEQ ID NO: 28 - DNA, 19 bp, synthetic construct
  misc_feature 1..19: note = Zic1 Anti-Sense Primer
  source 1..19: mol_type = other DNA
cacgtgcatgtgcttcttg

(19) United States
(12) Patent Application Publication
(10) Pub. No.: US 2024/0200036 A1
(43) Pub. Date: Jun. 20, 2024

(54) METHOD OF MAKING IN VIVO HUMAN SMALL INTESTINE ORGANOIDS FROM PLURIPOTENT STEM CELLS

(71) Applicant: Children's Hospital Medical Center, Cincinnati, OH (US)

(72) Inventors: James M. Wells, Cincinnati, OH (US); Carey Lane Watson, Cincinnati, OH (US); Jorge Orlando Munera, Cincinnati, OH (US); Maxime Mickael Mahe, Cincinnati, OH (US); Michael A. Helmrath, Cincinnati, OH (US); Michael J. Workman, Santa Monica, CA (US)

(21) Appl. No.: 18/156,726

(22) Filed: Mar. 8, 2024

Related U.S. Application Data
(63) Continuation of application No. 15/515,840, filed on Mar. 30, 2017, now Pat. No. 11,584,916.
(60) Provisional application No. 62/065,131, filed on Oct. 17, 2014.

(51) Int. Cl. C12N 5/071; A01K 67/0271; G01N 33/50

(52) U.S. Cl. CPC C12N 5/0679; A01K 67/0271; C12N 5/0697; G01N 33/5073; A01K 2227/105; A01K 2227/30; A01K 2267/03; C12N 2501/10; C12N 2501/11; C12N 2501/119; C12N 2501/155; C12N 2501/16; C12N 2501/345; C12N 2501/385; C12N 2501/415; C12N 2501/727; C12N 2502/08; C12N 2502/23; C12N 2503/00; C12N 2506/02; C12N 2506/45; C12N 2513/00

(57) ABSTRACT

Disclosed are methods for making a vascularized hollow organ derived from human intestinal organoid (HIOs). The HIOs may be obtained from human embryonic stem cells (ESC's) and/or induced pluripotent stem cells (iPSCs), such that the HIO forms mature intestinal tissue. Also disclosed are methods for making a human intestinal tissue containing a functional enteric nervous system (ENS).

### PRIORITY

This application is a continuation application of U.S. application Ser. No. 15/515,840, filed Mar. 30, 2017, which is a U.S. National Stage application under 35 U.S.C. § 371 which claims priority to and benefit of PCT/US2015/055956, filed Oct. 16, 2015, which in turn claims priority to and benefit of U.S. Provisional Application Ser. No. 62,065,131 titled “In vivo Model of Human Small Intestine Using Pluripotent Stem Cells,” filed Oct. 17, 2014, the contents of each are incorporated by reference in their entirety for all purposes.

### GOVERNMENT SUPPORT CLAUSE

This invention was made with government support under DK083325, DK098350, NS080815, DK092456, TR000546 and DK103117. The government has certain rights in the invention

### REFERENCE TO SEQUENCE LISTING

The present application is being filed along with a Sequence Listing in electronic format. The Sequence Listing is provided as a file entitled “CHMC63_006C1_Sequence_Listing.xml” created on Jan. 17, 2023, which is 35.70 KB in size. The information in the electronic format of the Sequence Listing is incorporated herein by reference in its entirety.

### BACKGROUND OF THE INVENTION

Because of the complexity of a vascularized, hollow organ such as the intestine, the development of an adequate human model for its study and replacement following surgery or pathological processes has proven to be a seemingly impossible task. Methods for studying the human intestine have largely required in vitro culture systems or have relied on animal models to address numerous translational questions, which do not always translate well in human studies. Traditional intestinal epithelial primary culture techniques were mostly limited to tissue culture technologies, such as organ cultures or intestinal cell lines that do not recapitulate the hierarch of stem cells to differentiated cells. While the recent identification of intestinal stem cells and conditions appropriate for human epithelial culture has overcome many of these obstacles, successful in vivo engraftment of epithelia cultures remains challenging because of the need for a supporting mesenchyme as exists in models exposing host mesenchyme following mucosal injury.

Differentiation of human pluripotent stem cells (hPSCs) into orga-specific subtypes offers an exciting avenue for the study of embryonic development and disease processes, for pharmacologic studies and as a potential resource for therapeutic transplant. To date, limited in vivo models exist for human intestine, all of which are dependent upon primary epithelial cultures or digested tissue from surgical biopsies that include mesenchymal cells transplanted on biodegradable scaffolds.

There is presently a need in the art for methods of making vascularized, hollow organs such as the intestine, in particular a model for the study of enteric nervous system (ENS) intestinal biology. Further, there is a need in the art for methods of making intestinal tissues having a functional enteric nervous system. Currently, methods for studying the human intestine have largely required in vitro culture systems or have relied on animal models. These studies, however, do not always translate well into human studies. While intestinal stem cells and human epithelial culture has addressed some of these problems, successful in vivo engraftment of epithelial cultures remains challenging because of the need for a supporting mesenchyme.

### BRIEF SUMMARY OF THE INVENTION

Disclosed are methods for making a vascularized hollow organ derived from human intestinal organoid (HIOs). The HIOs may be obtained from human embryonic stem cells (ESC's) and/or induced pluripotent stem cells (iPSCs), such that the HIO forms mature intestinal tissue. Also disclosed are methods for making a human intestinal tissue containing a functional enteric nervous system (ENS).

### BRIEF DESCRIPTION OF THE DRAWINGS

FIGS. 1A-1E. HIOs engraft in vivo to form mature intestinal tissue. FIG. 1A—Schematic representing development of HIOs from hPSCs and transplantation under kidney capsule to produce mature human intestinal tissue. FIG. 1B—Two HIOs in vitro at 35 d consisting of intestinal epithelium (black arrowheads) surrounded by supporting mesenchyme (white arrowheads). Scale bar, 100 μm. FIG. 1C—Engraftment (outlined) after 6 weeks with complex structure and established peripheral capillary network. The mouse kidney is seen below the engraftment for size comparison. Scale bar, 5 mm. FIG. 1D—Cross-section of engraftment at 6 weeks revealing intestinal structure with central lumen. Scale bar, 5 mm. FIG. 1E—Magnified luminal surface of engraftment displaying sheet of villi each with its own central capillary. Scale bar, 500 μm. n=139 transplants.

FIGS. 2A-2I. Engrafted intestinal tissue resembles adult intestine and is almost entirely of human origin. FIG. 2A—Low- and high-power imaging following H&E staining of engrafted HIO. Low magnification imaging demonstrates multiple areas of epithelium, laminated layers of smooth muscle and peripheral capillaries. Scale bars, 500 μm. High magnification imaging demonstrates crypt-villus domains as well as appropriate layers of subepithelium including lamina propria, muscularis mucosa, submucosa and outer smooth muscle layers. FIG. 2B—Alcian blue-periodic acid-Schiff staining of epithelium within engraftment revealing secretory lineages within the crypt-villus axis. Black arrowhead points to PAS-labeled Paneth cells present within crypt bases. FIG. 2C—All four intestinal lineages were present in engraftments including enterocytes (VIL), goblet cells (MUC2), Paneth cells (LYSO; white arrowhead; scale bars, 50 μm) and enteroendocrine cells (CHGA). E-cadherin (ECAD) was used for additional epithelial staining. FIG. 2D—Tuft cells are also seen throughout the epithelium, as labeled with doublecortin-like kinase 1 (DCLK1). FIG. 2E—mMECA-32 staining of mouse host vasculature ingrowth. FIG. 2F—Edu staining of active proliferation within crypt bases and proliferative zones within crypts of epithelium. FIG. 2G—Staining for VIM reveals the contribution of supporting mesenchyme, including laminated smooth muscle (white arrowheads) with staining of α-SMA. Merged images show dual staining with VIM and α-SMA, revealing a pericryptal sheath of supporting ISEMFs. FIG. 2H-2I—Contribution of human epithelial cells (FIG. 2H) and human mesenchymal tissue (FIG. 2I), as assessed by HuNuc staining through the full thickness of the engraftment. Dotted line separates engraftment from mouse kidney below. All scale bars are 100 μm except where specified otherwise. n=134 transplants.

FIGS. 3A-3I. Engrafted tissue matures in vivo and resembles mature small intestine. (FIG. 3A-D) Immunostaining of engrafted intestinal tissue (in vivo) revealing maturity of brush-border enzymes including SIM (FIG. 3A), DPPIV (FIG. 3B), LCT (FIG. 3C) and the differentiated enteroendocrine cell subtype (GIP) (FIG. 3D). ECAD and CDX2 were used for additional epithelial staining. (FIG. 3E-H) Staining of HIOs in vitro at comparable time points to transplants for SIM (FIG. 3E), DPPIV (FIG. 3F), LCT (FIG. 3G) or GIP for comparison (FIG. 3H). FIG. 3I. Relative gene expression of LCT, SIM, DPPIV and GIP in HIOs in vitro as compared to transplanted (Txp) HIOs. Values in graphs represent mean±s.e.m.; *P<0.05; **P<0.01; t-test. HIOs in vitro: n=4; transplants (Txp): n=8. Scale bars, 100 μm.

FIGS. 4A-4I. Engrafted human intestinal tissue responds to humoral factors following ileocecal resection (ICR) in the mouse host. (a) Schematic representing resection experiments in mice with transplanted HIOs. FIG. 4B. H&E staining of murine epithelium in sham versus ICR groups. Comparison of measured villus height (μm; FIG. 4C) and percentage of crypt fission (FIG. 4D) in mouse intestine between sham and ICR groups. (FIG. 4E) H&E staining of engrafted HIO epithelium in sham versus ICR groups. Comparison of villus height (FIG. 4F) and percentage of crypt fission (FIG. 4G) within engrafted HIOs in sham group versus ICR groups. (FIG. 4H) Immunofluorescence staining using Edu as a marker of intestinal cell proliferation in sham and ICR groups. ECAD is used to stain the epithelium. (FIG. 4I) Comparison of proliferative index (%) between sham and ICR groups in engrafted HIOs where proliferative index=number of Edu+ cells divided by total number of cells within intestinal crypt. Scale bars, 100 μm. Values in graphs represent mean±s.e.m. *P<0.05; **P<0.01; ***P<0.001; t-test. Sham group: n=4; ICR group: n=8.

FIGS. 5A-5F. Timeline/Schematic of HIO production, Size and Engraftment Efficiency. (a) Schematic representing directed differentiation of HIOs from hPSCs over a period of 35 days. FIG. 5B. Picture of HIO prior to transplant revealing central epithelium and surrounding supporting mesenchyme (outlined). FIG. 5C. Relative size of one HIO (outlined) embedded in type I collagen prior to transplant. FIG. 5D. Creation of pocket under kidney capsule using fine forceps. FIG. 5E. Picture of transplanted HIO under kidney capsule. FIG. 5F. Table showing efficiency of engraftments at 6 week time point after transplantation.

FIGS. 6A-6G. HIOs from induced pluripotent stem cells engraft to form mature human intestinal tissue in vivo. FIG. 6A. Engraftment 6 weeks after transplant using HIO derived from induced pluripotent stem cells. FIG. 6B. Magnified image of engraftment revealing luminal surface of engraftment with villi and central capillaries. FIG. 6C. Magnified H&E of epithelium within engraftment (Scale bar 100 μm). Crypt-villus domains were present as well as appropriate layers of sub-epithelium including lamina propria, muscularis mucosa, submucosa, and laminated outer smooth muscle layers. (FIG. 6D-G) All 4 intestinal lineages were present in engraftments including enterocytes (Villin—VIL) (Scale bar 100 μm) (FIG. 6D), Goblet cells (Mucin—MUC2) (FIG. 6E), Paneth cells (Lysozyme—LYSO) (FIG. 6F), and enteroendocrine cells (Chromogranin A—CHGA) (FIG. 6G). E-cadherin (ECAD) or CDX2 were used for additional epithelial staining. (All scale bars 50 μm except where specified).

FIGS. 7A-7D. Transmission Electron Microscopy (TEM) of engrafted tissue at 6 weeks. FIG. 7A. TEM image of brush border microvilli on the surface of intestinal epithelium. Tight and adherens junctions can also be seen in this image near the apical surface (scale bar 500 nm). FIG. 7B. TEM image of goblet cell with secretory granules (white) containing mucin (scale bar 2 μm). FIG. 7C. TEM image of enteroendocrine cell with secretory granules (dark) ready for release on basolateral aspect of cell (scale bar 2 μm). FIG. 7D. TEM image of smooth muscle within engraftment with parallel orientation of smooth muscle fibers (scale bar 10 μm).

FIGS. 8A-8I. Additional epithelial and mesenchymal markers of maturity in transplanted HIOs vs HIOs in vitro. (FIG. 8A-G) Comparison of relative gene expression of epithelial markers including EpCAM (FIG. 8A), Villin (VIL) (FIG. 8B), Alkaline phosphatase (ALPI) FIG. 8C, Lysozyme (LYZ) (FIG. 8D), Chromogranin A (CHGA) (FIG. 8E), Mucin (MUC2) (FIG. 8F), and Glucose transporter 2 (GLUT2) (FIG. 8G). (FIG. 8H-I) Relative expression of smooth muscle markers Desmin (DES) (FIG. 8H) and smooth muscle actin (α-SMA) (FIG. 8I). Values in graphs represent Mean±s.e.m. ns, not significant. *, P<0.05; **, p<0.01; ***, p<0.001; HIOs in vitro: n=4; Transplants (Txp): n=8.

FIGS. 9A-9K. Engrafted tissue displayed a mature epithelium maintained by intestinal stem cells. (FIG. 9A-D) An LGR5R5:eGFP BAC reporter ES line has been established and was used to generate HIOs expressing LGR5-eGFP cells FIG. 9A. LGR5-eGFP cells (green) were seen within an HIO in vitro scattered throughout the epithelium (FIG. 9B) and are localized in proliferative crypt base cells (colocalization with KI67), as expected in engrafted human intestinal tissue (FIG. 9C-D). FIG. 9E. Immunofluorescence staining of stem cell marker ASCL2 (green) localized within the crypt bases of the engraftment. FIG. 9F. Graph demonstrated relative fold changes of LGR5 and ASCL2 within HIOs in vitro prior to transplant and in engraftments. FIG. 9G. Enteroids were generated from the engrafted epithelium to demonstrate the stemness of the tissue. FIG. 9H. Panel shows Engraftment-derived epithelial enteroids following initial plating at days 1, 5, and 10 and after passaging. (FIG. 9I-K). Immunofluorescence staining revealed the presence of epithelial cells with Villin (VIL) and E-cadherin (ECAD) (FIG. 9I) as well as the presence of Paneth cells (Lysozyme—LYSO) (FIG. 9J) and brush border enzyme (Dipeptidyl peptidase 4—DPPIV) (FIG. 9K) (All scale bars 50 μm).

FIGS. 10A-10G. Engrafted intestinal tissue received its blood supply from the ingrowth of murine vasculature. FIG. 10A. A panendothelial antibody specific to mouse (mMECA-32) is used to reveal murine vasculature just below the surface of an engraftment (FIG. 10B) as well as within the interior of an engraftment (white arrowheads; Scale bars 50 μm). FIG. 10C. Magnified immunofluorescence image of mMECA-32 stained endothelium (green) comprising the vasculature within each villi as well as the capillary plexus just beneath the epithelium within the engraftment. FIG. 10D. Whole mount staining of engraftment within the kidney following FITC-labelled tomato lectin injections via mouse tail vein. Outlined areas 1 and 2 correspond with following images revealing functional fluorescent murine vasculature within the engraftments (Scale bars 50 μm). FIG. 10E. Immunofluorescence image revealing murine-specific (mMECA-32) and human specific (hCD31) staining of endothelium within an engraftment. FIG. 10F. Confocal imaging on whole mount engraftments did not showed connections between the murine and human vasculature. FIG. 10G. A murine specific marker of lymphatic vessels (LYVE-1) demonstrated ingrowth of lymphatic vessels from the murine host. (All scale bars 100 μm except where specified).

FIGS. 11A-11F. NSG Mice also undergo intestinal adaptation following surgical resection as well as the HIO graft. FIG. 11A. Magnified image of crypt fission (outlined) in murine small intestine following resection. (Scale bar 50 μm). FIG. 11B. Increased thickness of smooth muscle layer (tunica muscularis) in postoperative ICR tissue (Scale bar 50 μm). FIG. 11C. Comparison in murine intestinal crypt depth (μm) between Pre-operative, sham, and resected (ICR) groups. FIG. 11D. Comparison of thickness of smooth muscle layer (μm) in Pre-operative, sham, and resected (ICR) groups. FIG. 11E. Magnified image of intestinal epithelium in HIO graft in a sham mouse (Scale bar 50 μm). FIG. 11F. Magnified image of crypt fission (outlined) in HIO graft in an ICR mouse (Scale bar 50 μm). Values represented in graphs represent Mean±s.e.m. ns, not significant. *, p<0.05; **, p<0.01; ***, p<0.001; Pre-op group: n=13; sham group: n=10; ICR group: n=11.

FIGS. 12A-12C. Engrafted human intestinal tissue retained digestive and absorptive function. FIG. 12A. Intestinal alkaline phosphatase demonstrated activity ex vivo (Scale bar 100 μm). FIG. 12B. FITC-Dextran (MW 4,400) injected into engraftments in vivo (pictures seen before and after) increased significantly in murine serum from initial timepoint of 30 minutes compared with timepoint of 4 hours within each mouse (n=7). Values represented in graph represent Mean±s.e.m.; ns, not significant; *, p<0.05; **, p<0.01; ***, p<0.001; paired t-test. FIG. 12C. D-Ala-Leu-Lys-AMCA peptide uptake assay revealing peptide transporter (PEPT1) staining within the epithelium of an engraftment (scale bar 100 μm) FIG. 12C (i). Additional images showing fluorescence within the epithelium following injections of vehicle (DMEM solution), D-Ala-Leu-Lys-AMCA (DMEM+labeled peptide solution), and Captopril+D-Ala-Leu-Lys-AMCA (DMEM+labeled peptide+competitive inhibitor of transport solution) (n=3 for each group). Fluorescence was observed following injection of labeled peptide (iii), but little to no fluorescence was observed with either vehicle injection (ii) or with labeled-peptide+captopril injection (iv).

FIGS. 13A-13D. Quality control assays for iPSCs. FIG. 13A. Phase contrast image of iPSC colonies cultured in standard feeder free conditions. FIG. 13B. Immunofluorescence staining of pluripotency markers Oct4 and Nanog in iPSCs. FIG. 13C. G-banded karyotype analysis demonstrating normal (46,XY) karyotype of control iPSCs. FIG. 13D. H&E stained sections of a teratoma derived from iPSCs showed tissue arising from the three embryonic germ layers, endoderm, ectoderm and mesoderm. Control teratomas presented stellate reticular cells surrounded by peripheral epithelium with anti-basal nuclei consistent with primitive tooth (ectoderm), ciliated columnar epithelium consistent with intestine (endoderm), and cartilage cells (mesoderm). The images are 400-fold magnification.

FIGS. 14A-14D. Generating Vagal-like Neural Crest Cells (NCCs). 14A—Schematic representation of protocol for generating neural crest cells expressing Hox genes. While this method has been reported to generate cranial NCCs 9 addition of retinoic acid during the last 48 hours of neurosphere culture induces Hox A2, B3, B5 and B7, markers of posterior/vagal-like NCCs. FIG. 14B—NCCs displayed stellate morphology and were positive for surface markers HNK-1, p75NTR, and RET. FIG. 14C—Quantitative RT-PCR analysis for of neural plate border and regulators of neural crest cell specification. FIG. 14D—Treatment of neurospheres for 48 hours with RA results in the formation of NCCs that express Vagal-level HOX genes. The more posterior HOX genes B3, 5, and 7 showed dose-dependent increase in levels of expression (data not shown). Scale bars, 100 μM. Values in graphs represent mean±s.e.m.; *P<0.01, **P<0.001; Student's t-test (two-tailed, unpaired); n=3 biological replicates per condition; data representative of 3 independent experiments.

FIGS. 15A-15C. Incorporation of NCCs into developing HIOs in vitro. FIG. 15A—Schematic for incorporating NCCs into HIOs. HIOs and NCCs were generated separately, combined by low-speed centrifugation, embedded in Matrigel and grown for 4 weeks in vitro. In some cases, HIOs were transplanted into NSG mice and grown in vivo for 6-8 weeks. FIG. 15B—In vitro growth of HIOs. Left panel—Bright-field images of 28d HIOs+/−NCCs. Scale bar, 1 mm. Middle panel—Immunostaining for neurons (βIII-tubulin) and epithelium (E-cadherin). Right panel—Immunostaining for glial cells (S100+) and epithelium (E-cadherin). Scale bar, 100 μm. Data is representative of 14 independent experiments combining HIOs with NCCs in vitro. FIG. 15C—In vivo growth of HIOs+NCCs. HIOs±NCCs were transplanted into the kidney subcapsular space of NSG mice and grown for 6 weeks. Left panels show HIOs±NCCs that were ˜1 cm in diameter. Scale bar, 1 mm. Middle panels show the formation of neurons adjacent to smooth muscle fibers (Desmin+) in a myenteric-like position, oriented perpendicular to one another in HIOs−NCC, demonstrating that smooth muscle development occurs in the absence of an ENS. A second layer of desmin+ cells is located submucosally to the epithelium. HIOs+NCCs contain neurons (BIII-tubulin+) that are embedded within the Desmin+ smooth muscle layers and have an organization that closely resembles a myenteric plexus. Right panels show that glial cells (S100+) are also embedded within the mesenchymal layers of HIOs+NCCs, including the submucosal layer.

FIGS. 16A-16B. Neuronal diversity and formation of interstitial cells of Cajal (ICC). FIG. 16A. Analysis of different neuronal cell types using neurochemical markers of ENS neurons in HIOs+NCCs cultured in vitro (upper panels) and following engraftment in vivo (lower panels). Dopaminergic neurons (TH), interneurons (ChAT, 5-HT), sensory neurons (Calbindin), excitatory neurons (Calretinin) and inhibitory neurons (nNOS) were all found in in vivo engrafted HIOs+NCCs. In contrast, in vitro HIOs+NCCs did not contain inhibitory neurons (nNOS), suggesting that they were embryonic in nature. FIG. 16B. Formation of interstitial cells of Cajal (CD117—red) in HIOs+NCCs in vivo. HIOs without NCCs did not form neurons (BIII-tubulin—green) and had fewer CD117+ cells, suggesting that differentiation of ICCs may involve NCCs.

FIGS. 17A-17D. Live imaging of neural activity in HIOs+NCCs. FIG. 17A. Schematic representation of protocol for combining NCCs generated from GCaMP6f expressing PSCs with HIOs generated from H1 PSCs. FIG. 17B. Live imaging of Ca2+ flux in the neural crest derived ENS cells shows periodic activity. Snapshots from a 20 min time-lapse movie showing neural activity in HIOs+NCCs. FIG. 17C. Numbered snapshots show the rhythmic waves of depolarization of single neurons in HIO+NCC cultures (only the first 3 time points were shown in FIG. 17B). The graph measures pixel intensity related to Ca2+flux. FIG. 17D KCl induces wave of Calcium efflux. Quantification of pixel intensity from KCl experiment shows that KCl induces a rapid and broad wave of depolarization of ENS cells.

FIGS. 18A-18C. Formation of a 3-dimensional submucosal and myenteric plexus that controls motility. FIG. 18A Whole mount immunostaining and 3-D imaging of human intestine and HIO+ENS tissues. En face view of human submucosa and HIOs+ENS, showing arrangement of neurons (BIII-tubulin) and glia (S100) into a neuroglial plexus that integrates and orients with smooth muscle fibers (desmin). HIO+ENS tissues also contained a neuroglial plexus that oriented with smooth muscle fibers. FIG. 18B—Formation of an enteric and submucosal plexus. Left panel—en face view of in vivo engrafted HIO+NCCs shows close association of glia (S100) around the epithelium (dapi—blue). Right panel—a lateral view of whole-mount images clearly identifies two plexuses; one associated with the epithelial mucosa (submucosal plexus) and the other associated with outer layers of smooth muscle (myenteric plexus). FIG. 18C The ENS in HIOs mediates peristaltic-like contractions. In vivo grown tissues were explanted into Tyrode's solution and subjected to an electrical field stimulation (EFS). HIO−ENS that were subjected to a high voltage EFS (1 ms pulse at 100V) had one single contraction (n=2). HIO+ENS that were subjected to a low voltage EFS (1 ms pulse at 50V) had a sustained series of wave-like contractions (n=5) that were lost when tissues were cultured in the tetrodotoxin (HIO+ENS+TTX, n=2).

FIGS. 19A-19E. ENS-dependent and independent control of contractile activity. FIG. 1A Recordings of spontaneous contractions in transplanted HIOs and HIO+ENS tissue strips. Phasic contractions were observed after tissue equilibration (no stimulation) suggestive of interstitial cells of Cajal (ICCs) present in both HIO (n=7) and HIO+ENS (n=7) tissues. FIG. 19B—Recordings showing an inhibition of ICCs activity with Methylene Blue leading to loss of contractile activity (n=3). FIG. 19C—Left panel shows representative images of Dimethylphenylpiperazinium (DMPP) stimulation in HIO and HIO+ENS. Right panel represents area under the curve (AUC) during DMPP (10 μM) stimulation measured for 2 minutes before and after stimulation (n=7). FIG. 19D—TTX inhibition of ENS activation. Area under the curve during DMPP stimulation measured for 2 minutes after stimulation, followed by tetrodotoxin (TTX; 10 μM) treatment in HIO+ENS (n=7). FIG. 19E—ENS-induced relaxation by a NO-dependent mechanism. Area under the curve during DMPP stimulation measured for 2 minutes after stimulation, followed by NG-nitro-L-arginine methyl ester (L-NAME) treatment in HIO+ENS (n=7). Values in graphs represent mean±s.e.m.; *P<0.05, **P<0.01; Mann & Whitney test.

FIGS. 20A-20B. Characterization of developing neural crest cells. FIG. 20A. Day 6 free-floating neurospheres are roughly 500 μm in diameter and positive for PAX6, Vimentin, and SOX9; characteristic of neural stem cells and the lateral neural plate where neural crest cells form. FIG. 20B. Neurospheres attached to a fibronectin substrate and formed neural rosettes visible with bright field microscopy. Attached neurospheres broadly expressed PAX6 whereas Vimentin staining was observed at the edge of neural rosettes where NCCs were delaminating. As NCCs migrated away from rosettes, PAX6 was down regulated whereas SOX2 expression was maintained.

FIGS. 21A-21F. Comparison of human and chick NCC behavior. (FIG. 21A and FIG. 21B) Isolation and culturing of cranial (Hox-negative) and trunk (Hox-positive) NCCs from chick embryos and analysis of Hox gene expression. NCCs isolated from anterior chicken neural tube do not express Hox genes while those isolated from posterior neural tube are HoxB7 positive. (FIG. 21C) Chicken cranial NCCs express vagal-level Hox genes HoxB3 and HoxB7 when treated with 2 μM RA for 48 hours. A different posterizing factor, FGF4, had no effect on Hox gene expression. (FIG. 21D) Schematic describing engraftment of human PSC-derived NCCs into chicken embryos. GFP-labeled human NCCs were injected intersomitically into HH10-12 chick embryos in ovo. (FIG. 21E) Embryos were analyzed at ˜HH38. GFP-labeled cells had migrated laterally along the neural tube (NT) and could be found colonizing the foregut (FG). (FIG. 21F) Differentiation of GFP-labeled cells into peripheral neurons (peripherin) (dorsal, right; anterior, up).

FIGS. 22A-22B. Differentiation potential of cranial and RA-treated NCCs. FIG. 22A. In vitro differentiation of human PSC-derived NCCs into neuroglia lineages and mesenchymal lineages (osteocytes and chondrocytes). Cranial and RA-treated NCCs were equally competent to form neuroglial lineages (peripherin and GFAP), osteocytes (alizarin red) and chondrocytes (alcian blue) in vitro. FIG. 22B. Cranial-like NCCs formed pigmented epithelium (arrowhead) in vitro and in vivo, suggesting a retained competence to form neuroepithelium.

FIGS. 23A-23C. Generation of Human Intestinal Organoids (HIOs) and expression of NCC migratory cues. FIG. 23A. Method for generating HIOs through directed differentiation of human pluripotent stem cells. FIG. 23B. Temporal expression of differentiation markers at each stage of HIO development. Activin A mediated efficient differentiation into definitive endoderm (FOXA2 and SOX17), WNT activation in combination with FGF4 induced CDX2 expression in monolayers and free-floating spheroids. Growth of spheroids in Matrigel for 4 weeks resulted in the formation of HIOs expressing CDX2. HIOs were engrafted into the subcapsular space of mice, where after 6 weeks they grew and matured to form intestinal tissue with crypts and villi. The human-specific antibody HuNu shows that intestinal epithelium and mesenchyme are human in origin. FIG. 23C. HIOs formed in vitro expressed GDNF and EDN3. Values in graphs represent mean±s.e.m.; *P<0.05, **P<0.01; t-test (two-tailed, unpaired); n=3 (biological replicates); data representative of 2 independent experiments.

FIG. 24. Neurons and glia in HIOs+NCC cultures are NCC-derived. NCCs were generated from H9-GAPDH-GFP human embryonic stem cells, which ubiquitously express GFP, and combined with HIOs generated from H1 HESCs. HIOs were co-stained for GFP and pan-neuronal marker PGP9.5 or the glial marker S100. Neuroglial cells in HIOs+NCCs are NCC-derived as is evidenced by co-expression of GFP and neuroglial markers.

FIGS. 25A-25B. Ganglia-like structures form in HIOs+NCCs grown in vivo. (FIG. 25A) In immunostained sections, Neurons (BIII-tubulin) were spread throughout the myenteric plexus and Applicant observed neuronal cell bodies (HuC/D) in clusters, similar to ganglia. (FIG. 25B) Whole mount immunostaining for neurons (BIII-tubulin, en face view) and neural cell bodies (HuC/D, lateral view) shows neuronal cell bodies organized into a ganglion-like clusters in HIOs+NCCs.

FIG. 26. Recordings of spontaneous contractions in transplanted HIOs and HIO+NCC tissues.

FIGS. 27A-27D. (FIG. 27A) Left panel shows representative recording of Veratridine stimulation in HIO and HIO+NCC. Right panel represents area under the curve (AUC) during Veratridine (3 μM) stimulation measured for 2 minutes before and after stimulation (n=5). (FIG. 27B) Area under the curve during Dimethylphenylpiperazinium (DMPP; 10 μM) stimulation measured for 2 minutes before and after stimulation, followed by tetrodotoxin (TTX; 10μM) treatment (n=3). (FIG. 27C) Area under the curve during DMPP stimulation measured for 2 minutes after stimulation, followed by NG-nitro-L-arginine methyl ester (L-NAME) treatment (n=3). (d) Left Panel representative recordings following Sodium Nitroprusside (SNP) stimulation of both HIO (n=4) and HIO+NCC tissues (n=14). Right Panel area under the curve during SNP stimulation measured for 2 minutes before and after stimulation. Values in graphs represent mean±s.e.m; Mann & Whitney test.

### DETAILED DESCRIPTION OF THE INVENTION

The enteric nervous system (ENS) of the gastrointestinal (GI) tract controls motility, epithelial permeability and fluid exchange. Perturbations in ENS development or function are common, yet a human model to study ENS-intestinal biology is lacking.

Applicant has generated human intestinal organoids (HIOs) produced in vitro from human embryonic stem cells (ESCs) or induced pluripotent stem cells (iPSCs) that can engraft in vivo. These HIOs form mature intestinal epithelium with intestinal stem cells contributing to the cryvillus architecture and a laminated human mesenchyme, both supported by mouse vasculature ingrowth. Applicant has shown that in vivo transplantation resulted in marked expansion and maturation of the epithelium and mesenchyme, as demonstrated by different intestinal cell lineages (enterocytes, goblet cells, Paneth cells, tuft cells, and enteroendocrine cells) presence of functional brush-border enzymes (lactase, sucrose-isomaltase and diptidyl peptidase 4), and visible subepithelial and smooth muscle layers when compared with HIOs in vitro. Applicant has further shown that transplanted intestinal tissues demonstrated digestive functions as shown by permeability and peptide uptake studies. Transplanted HIO-derived tissue was found to be responsive to systemic signals from the host mouse following ileocecal resection, suggesting a role for circulating factors in the intestinal adaptive response.

Applicant further has developed a human PSC-derived intestinal tissue with a functional ENS. Using a tissue engineering approach with pluripotent stem cells (PSCs) to generate human intestinal tissue containing a functional ENS, Applicant has recapitulated normal intestinal ENS development by combining PSC-derived neural crest cells (NCCs) with developing human intestinal organoids (HIOs). When cultured alone, Applicant has found that NCCs had full differentiation potential in vitro, however when recombined with HIOs they differentiated into neurons and glial cells. NCC-derived ENS neurons were found to self-assemble within the developing intestinal mesenchyme and exhibited neuronal activity as measured by rhythmic waves of calcium transients. ENS-containing HIOs grown in vivo formed neuroglial structures similar to a myenteric and submucosal plexus, formed interstitial cells of Cajal, and had an electro-mechanical coupling that regulated waves of propagating contraction. This is the first demonstration of a human PSC-derived intestinal tissue with a functional ENS.

The enteric nervous system (ENS) is essential for GI motility, secretion, blood flow, epithelial barrier permeability and fluid exchange. Developmentally, the majority of the ENS arises from vagal neural crest cells (NCCs) that derive from the dorsal neural tube and migrate ventrally to colonize the foregut around embryonic week 4 in humans. Subsequently, the NCCs extensively proliferate and migrate caudally to colonize the entire GI tract by 7 weeks of human gestation. When enteric NCCs fail to properly migrate, proliferate, survive, and/or differentiate in the GI tract, defects in the structure and function of the ENS result and patients present with a spectrum of enteric neuropathies. In addition enteric neuropathies are common in digestive diseases such as inflammatory bowel disease and often occur secondarily in diseases such as Parkinson's disease, diabetes mellitus and age-related degeneration. The lack of human ENS model systems for studying physiopathological processes of enteric neuropathies may account for the surprisingly slow progress in their diagnosis and treatment. The long-standing treatment of patients with congenital lack of enteric ganglia, Hirschsprung's disease (1:5000 births), involves surgical resection of the aganglionic gut segment leaving a greatly reduced normal bowel. Although the remaining bowel contains ganglia, it remains unclear why many of these patients suffer from recurrent bouts of enterocolitis and dysmotily. Enteric neurons derived from pluripotent stem cells (PSCs), enteric progenitors, or even CNS neural stem cells have been shown to incorporate and function in aganglionic chick and murine GI explants, suggesting that a similar approach might work therapeutically in humans.

Despite the progress towards cell-based clinical treatments much work remains to be done regarding our understanding of human ENS development and disease. Very little is known about the etiology of enteric neuropathies, and mechanisms driving various aspects of human ENS development such as formation of neuronal diversity remain unclear. Although there is currently no way to functionally study human ENS development, recent progress using the directed differentiation of human pluripotent stem cells has resulted in the formation of complex and physiological 3D human organ cultures called organoids. Organoid models have been developed for intestine, liver, stomach, CNS, thyroid, and lung, to name a few, and have allowed for unprecedented studies of human developmental biology and disease. Despite the remarkable complexity of organoids, they lack cell and tissue types that are required for full organ function. For example, none of the organoid systems contain an integrated peripheral nervous system.

As disclosed herein, Applicant has demonstrated the development of 3D human intestinal organoids (HIOs) containing a functional eteric nervous system. PSCs were first differentiated into vagal-like neural crest cells (NCCs) and then introduced into developing intestinal cultures at the stage corresponding to gut tube formation to approximate normal colonization of the embryonic intestine by the ENS. The resulting HIOs could be grown in vitro>8 weeks, and resembled developing fetal intestine, with a diverse set of neurons capable of rhythmic and stimulated waves of calcium transients. When transplanted in vivo, HIOs formed complex, mature intestinal epithelium with crypts and villi surrounded by submucosal and myenteric smooth muscle layers. HIOs with an ENS formed both a submucosal and myenteric neuroglial plexus. The plexus contained bundles of neural cell bodies with a network of interganglionated fibers that integrated into the layers of smooth muscle. Electrical field stimulation of in vivo grown HIOs with an ENS elicited waves of propagating motility that were blocked with the neurotoxin tetrodotoxin. Organ-bath studies of tissue strips further demonstrated a functional nitrergic neuro-muscular coupling in HIOs with ENS. Thus, Applicant's invention is believed to be the first evidence for in vitro generation of human intestinal tissue with a functional enteric neural system fully derived from human pluripotent stem cells.

In one aspect, a method of making a vascularized hollow organ is disclosed. In this aspect, the method may comprise the steps of a) engrafting a human intestinal organoid (HIO) into an immune compromised organism, for example a mammal, for example a mammal having no immune response, for example a mammal having severe combined immunodeficiency disorder (SCID). The HIO may be obtained from human embryonic stem cells (ESCs) and/or induced pluripotent stem cells (iPSCs). In one aspect, during the engrafting step, the HIO forms mature intestinal tissue.

In one aspect, the human intestinal organoid (HIO) may be embedded in collagen. In one aspect, the collagen may be type I collagen.

The engrafting step may include transplantation of the HIO into a kidney capsule of an immune compromised organism. The engrafting step may be carried out for a period of at least about three weeks, or at least about four weeks, or at least about five weeks, or at least about six weeks. The duration of time with regard to this step may be determined by one of ordinary skill in the art. The engraftment step may be carried out, for example, until the intestinal tissue meets one or more criteria. Such criteria may include, for example, having a columnar intestinal epithelium surrounded by a supporting mesenchyme, growth of 1-3 cm in diameter, the formation of villi and crypts containing functional intestinal cells, having submucosal and myenteric layers of smooth muscle fibers, or a combinations thereof.

In one aspect, a method of making a human intestinal tissue containing a functional enteric nervous system (ENS) is disclosed. In this aspect, the method may comprise the steps of a) contacting vagal-like neural crest cells (NCCs) derived from human ES cells and/or iPS cells (IPCs) with a three dimensional human intestinal organoid (HIO); and c) transplanting said HIO in vivo. In one aspect, the NCCs may be obtained by contacting human ES cells and/or iPS cells with retinoic acid. In this aspect, the retinoic acid may be contacted with the human ES cells and/or iPS cells in an amount sufficient to cause posteriorization. In some aspects, the retinoic acid may be contacted with the human ES cells and/or iPS cells for a period of about 1 to about 2 days, or in some aspects, about 2 days. The retinoic acid contacting step may be carried out for a period of about two days at the neurosphere stage, or until substantial expression of HOXB3, HOXb5, and/or HOXb7 is observed.

In one aspect, the transplanting step may be carried out for a period of time sufficient to allow detection of neurons and/or glia. In one aspect, the neurons may comprise BIII-tubulin. In a further aspect, the glia comprise S100. In a yet further aspect, the neurons and glia may integrate into smooth muscle layers (desmin+ cells). In one aspect, the transplanting step may be carried out for a period of time sufficient to allow formation of nNOS+ inhibitory neurons.

In one aspect, the human intestinal tissue containing a functional enteric nervous system (ENS) as described herein may be capable of contractile activity.

Further disclosed is a method of treating a patient requiring replacement of a portion of a gastrointestinal tract, which may comprise the step of replacing a portion of the patient's gastrointestinal tract with a human intestinal tissue manufactured according the methods as described herein. In a further aspect, disclosed is a method of determining the effect of a treatment on a human intestinal tract, comprising the step of contacting the treatment of interest with a human intestinal tissue manufactured according the methods as described herein. The model provided by Applicant may be useful for studies of intestinal physiology, disease, and/or translational studies.

### EXAMPLE

### Development of an in Vivo Model of Human Small Intestine Using Pluripotent Stem Cells

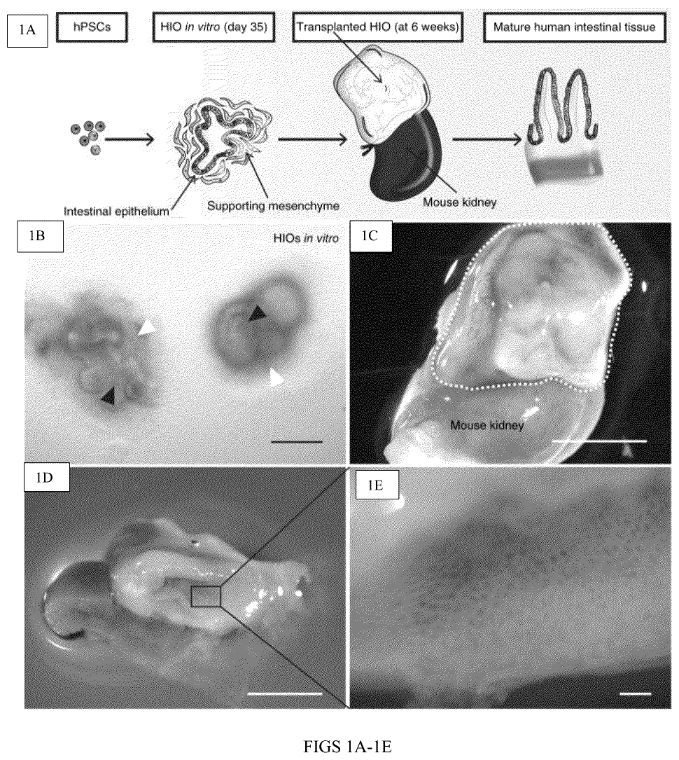

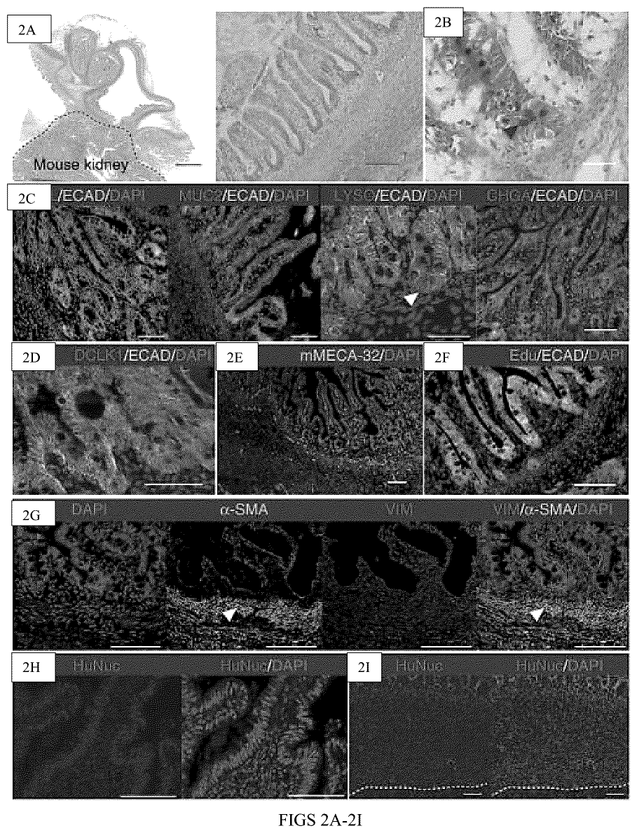

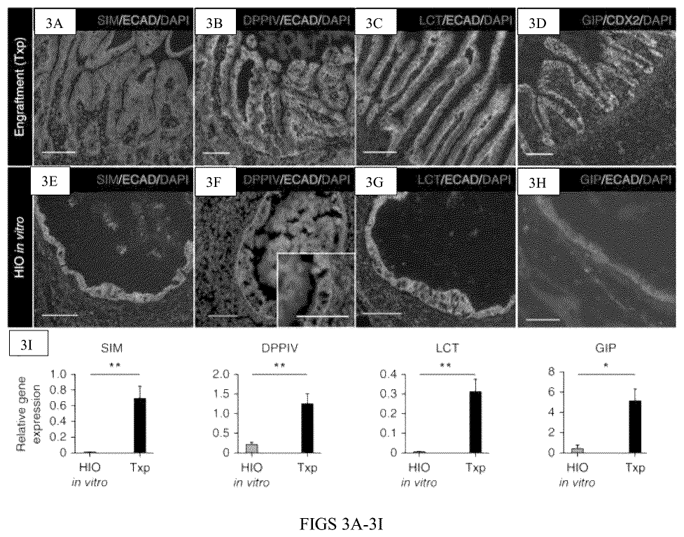

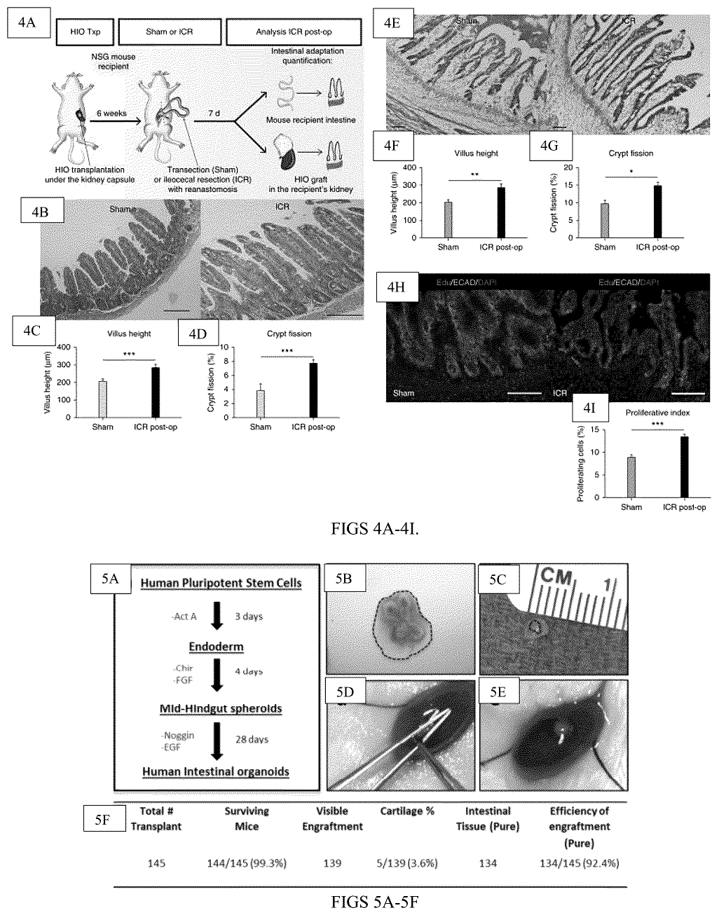

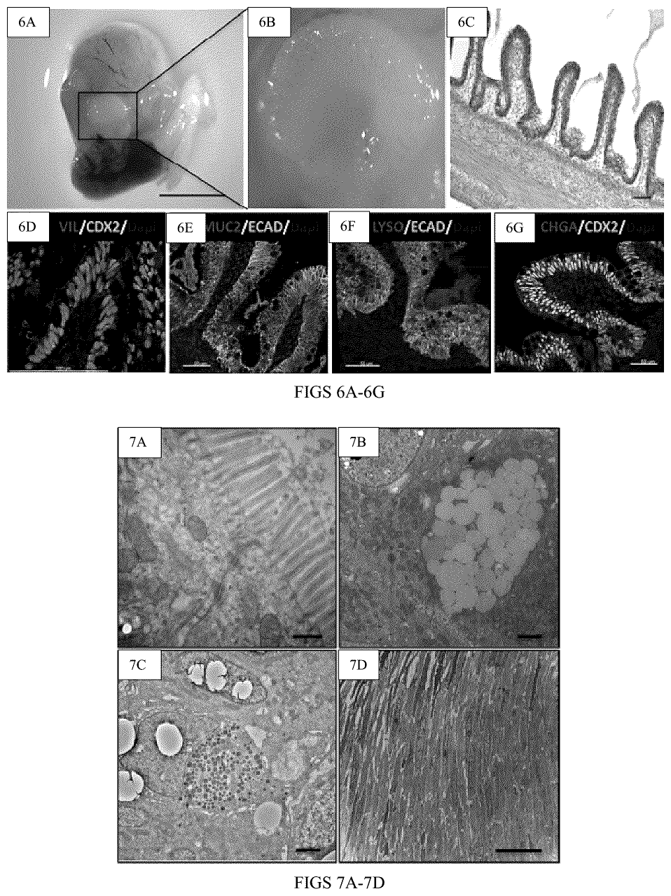

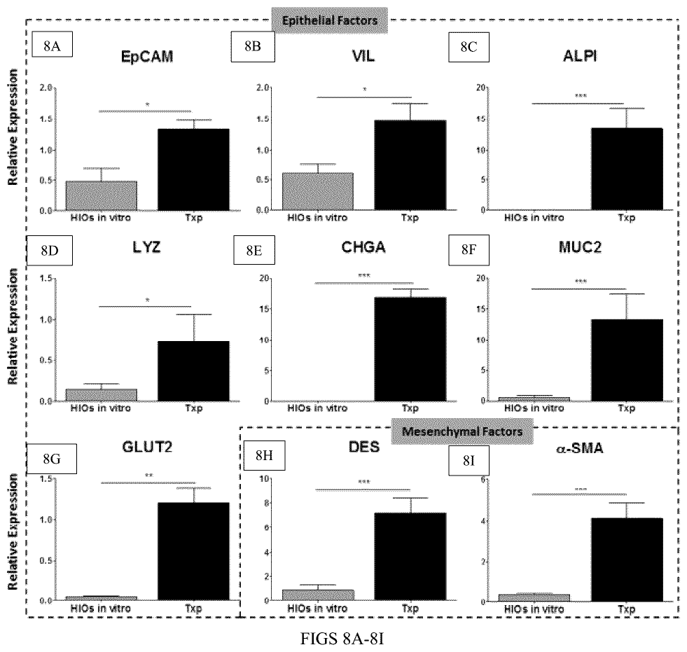

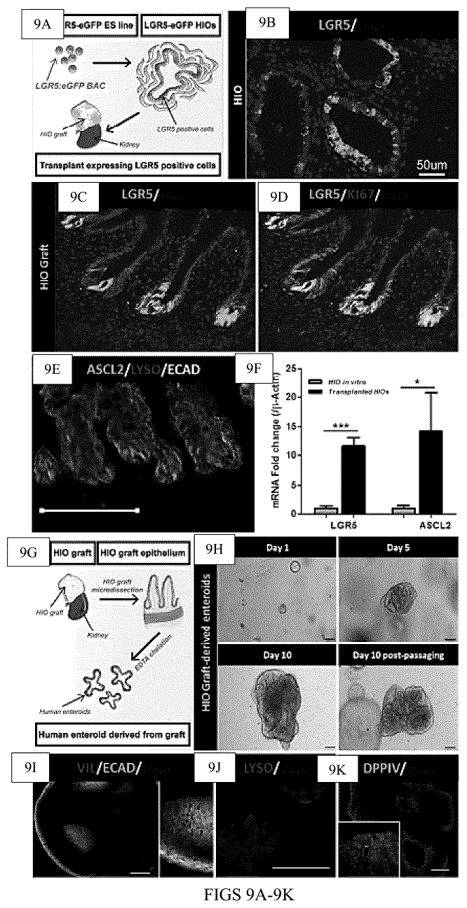

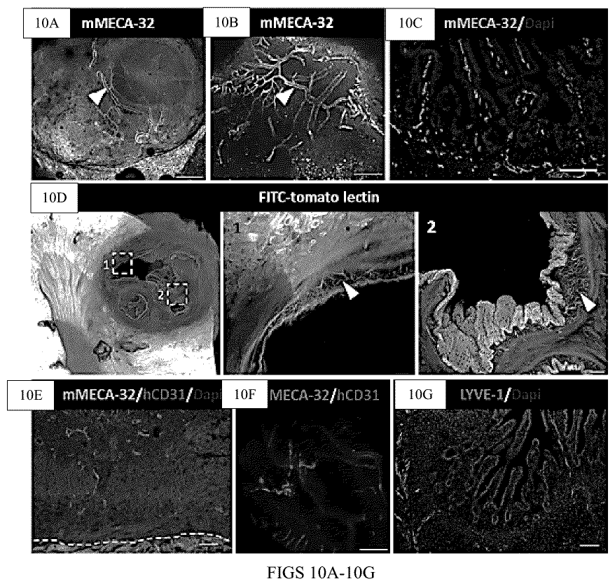

To establish an in vivo HIO model, Applicant generated HIOs from human ESCs or iPSCs as previous described (Spence, et al, Nature 2011; McCracken et al, Nat. Protoc. 2011). The differentiation process took approximately 35 days. (FIG. 1A and FIG. 5A) and produced HIOs with columnar intestinal epithelium surrounded by a supporting mesenchyme (FIG. 1B and FIG. 5B). Applicant then embedded the HIOs into type I collagen and transplanted them under the kidney capsule of immunocompromised nonobese diabetic severe combined immunodeficiency interleukin-2Rγ^{null} (NSG) mice and allowed to mature and grow for six weeks (FIG. 1A and FIG. 5D, 1E). At the time of harvest, the HIOs had grown considerably in size, upwards of 50- to 100-fold larger in volume (FIG. 1C, FIG. 1D and FIG. 5C and FIG. 6A) and were highly vascularized (FIG. 1C). Of note, 92.4% of the transplanted HIOs successfully engrafted under the kidney capsule (FIG. 5F). Visual inspection of tissues revealed intestinal morphology (FIG. 1D) with mucous-filled lumens and well-developed sheets of villi, each with its own central capillary network (FIG. 1E)). Histologically, this engrafted tissue resembled native human intestine with crypt-villus architecture and underlying laminated submucosal layers including lamina propria, muscularis mucosa, and submucosal and outer smooth muscle layers (FIG. 2A and FIG. 6C). When compared with their in vitro counterparts (data not shown), the engrafted tissue in vivo appeared more mature and differentiated, with all major intestinal cell lineages, including enterocytes, goblet cells, Paneth cells, eneroendocrine cells and tuft cells, located within appropriate regions of the crypt-villus axis (FIG. 2B-D) and FIG. 6D-G). Paneth cells were located within crypt bases as expected rather than scattered throughout the epithelium (FIG. 2B, 2C). Transmission electron microscopy (TEM) revealed a brush border with well-developed tight junctions similar to that of HIOs in vitro (FIG. 7A); however, mature goblet cells and enteroendocrine cells, as seen within the epithelium of engraftments (FIG. 7B, C), were not present in TEM of HIOs in vitro (data not shown). Within the epithelium, Applicant observed increased relative expression of genes characteristic of the epithelia cell types when comparing engraftments to HIOs in vitro (FIG. 8A-G). Applicant found that blood vessels within the engraftment stained positive for mouse-specific panedothelial cell antigen (mMECA-32) (FIG. 2E). The engrafted tissue was also actively proliferating within discreet crypts, as revealed by incorporation of 5-ethanyl-2′-deoxyuridine (Edu) (FIG. 2F). Using transgenic human LGR5 reporter HIOs to trace intestinal stem cells, Applicant found that actively proliferating labeled stem cells were present within the HIOs and localized to the base of the crypts in the engraftment (FIG. 9A-D). Additionally, the engrafted tissue expressed the stem cell marker ASCL2 (FIG. 9E-F), and Applicant was able to generate enteroids derived from the epithelium of engraftments, further demonstrating the existence of an intestinal stem cell pool (FIG. 9G-K). These data suggest that HIOs undergo considerable maturation into mature intestinal tissue following engraftment in vivo.

Cross-talk between the adjacent mesenchyme and intestinal epithelium is known to play a major role during GI development. It was previously shown that the supporting mesenchyme develops alongside the intestinal epithelium of HIOs in vitro and contains immature populations of subepithelial myofibroblasts, fibroblasts and smooth muscle cells. Applicant investigated whether HIO mesenchyme also developed into more mature, differentiated cell types following engraftment in vivo. The non-epithelial regions of the engrafted tissue stained positive for the mesenchymal marker vimentin (VIM) and included several laminated subepithelial layers, including distinct smooth muscle layers positive for a-smooth muscle actin (α-SMA), revealing the mesenchymal contribution of the engraftment (FIG. 2G). Additionally, dual staining for α-SMA and VIM revealed a pericryptal sheath (FIG. 2G) of intestinal subepithelial myofibroblasts (ISEMFs) which are known to support in vitro and in vivo growth of human small intestinal epithelial stem cells. Likewise, the intestinal epithelium has been shown to contribute appropriate signals for the development of the subepithelial mesenchymal layers including lamina propria, ISEMFs and muscularis mucosa leading to appropriate laminations of the subepithelium (FIG. 2A, 2G). In addition to the maturity of smooth muscle layers histologically, relative expression of desmin and α-SMA was increased in HIOs engrafted in vivo as compared to HIOs in vitro (FIG. 8H, 8I). TEM of the engraftments revealed smooth muscle cells with parallel orientation similar to that of adult intestine (FIG. 7D). These data are consistent with published reports that successful engraftment and maturation of human intestinal tissues requires the presence of a mesenchymal niche. For example, lack of a mesenchymal component has resulted in failure of engraftment, whereas including mesenchymal cells along with epithelium resulted in successful engraftment.

As our engraftments contained a variety of mesenchymal cell types, including endothelial cells, Applicant next investigated which components were of human origin and which were from the host. As expected, HIO epithelium was completely of human origin and stained positive for a human nuclear antigen (HuNuc) (FIG. 2H). In addition, the majority of tissue of mesenchymal origin, including lamina propria, muscularis mucosa, submucosa and smooth muscle layers, also stained positive for HuNuc (FIG. 2I). In contrast, the majority of blood vessels were of mouse origin as they stained positive for mMECA-32 (FIG. 2E). The blood supply of the engrafted HIO was supported by mouse vasculature, as demonstrated by positive labeling of endothelium with FITC-conjugated tomato lectin tail vein injection (FIG. 10A-10D). Few blood vessels of human origin were positive for human-specific panendothelial cell antigen in the engraftment, and these were not connected to the mouse vasculature (FIG. 10E, 10F). Lymphatic vessels were of mouse origin and stained positive for mouse-specific lymphatic vessel endothelial hyaluronan receptor 1 (LYVE-1) (FIG. 10G).

Engraftment of PSC-derived pancreatic progenitor cells has been shown to enhance development of islet cells in vivo. Applicant analyzed the impact of in vivo growth on the maturity of our engraftments at 6 weeks as compared to HIOs in vitro at a similar time point (35 d plus 6 weeks in vitro) by determining the expression of several markers of mature epithelium at both the mRNA and protein levels. Applicant observed marked increases in mRNA and/or protein expression of markers characteristic of differentiated enterocytes, including dipeptidyl peptidase 4 (DPPIV), glucose transporter type 2 (GLUT2), sucrose-isomaltase (SIM) and villin (VIL), compared to HIOs in vitro (FIG. 3A, 3B, 3E, 3F, 3A and FIG. 8B, 8G). Additionally, Applicant also looked at the brush border enzymes alkaline phosphatase (ALPI) and lactase (LCT), as well as markers of secretory cell types, including enteroendocrine (gastric inhibitory peptide (GIP) and chromogranin A (CHGA)), goblet (mucin 2 (MUC2)) and Paneth cells (lysozyme (LYSO)). For each of these markers, there were also marked increases in mRNA and protein expression in engrafted HIOs (FIG. 3C, 3D, 3G and FIG. 8C-8F). Therefore, Applicant concluded that in vivo growth promotes maturation of the intestinal tissue.

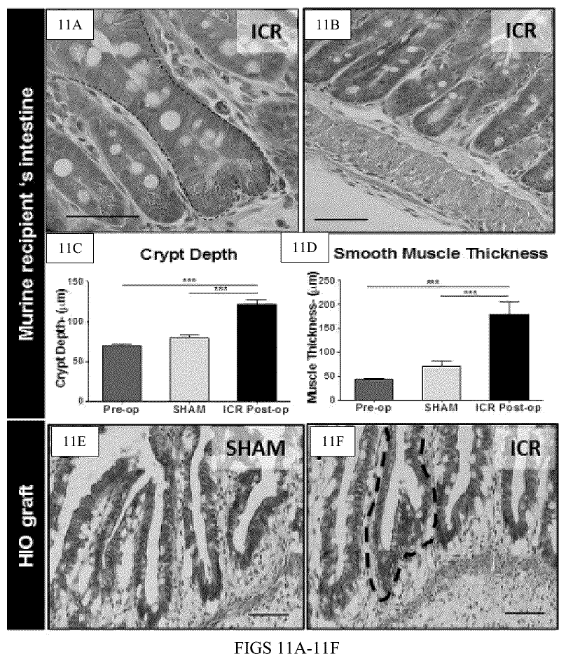

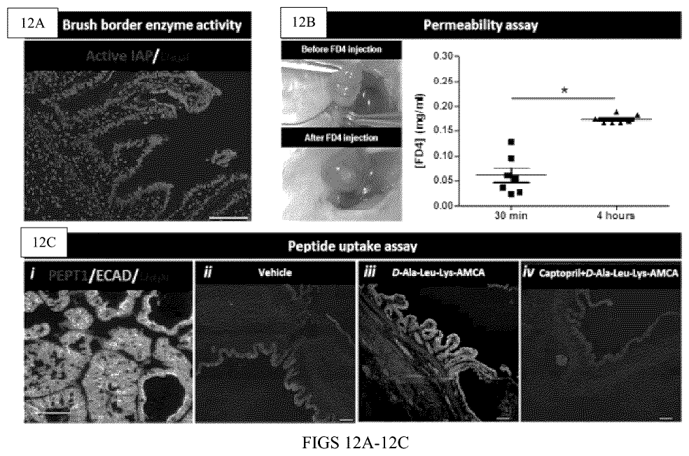

Given the mature phenotype of engrafted HIOs in vivo, Applicant postulated that these tissues might represent a new model to study in vivo physiology of the human gut. Applicant therefore investigated whether engrafted HIOs could respond to physiologic cues elicited by intestinal resection. Humoral factors have been suggested to be involved in the intestinal adaptive response. In rats connected in a parabiotic relationship via vascular anastomosis, surgical resection in one rat led to increased intestinal proliferation in the parabiotic partner 7. However, there is no model available to investigate this phenomenon with human intestine. Here, Applicant used a model of ileocecal resection (ICR) in the transplanted mice to investigate whether circulating humoral factors that are stimulated in response to intestinal resection could affect engrafted human intestinal tissues in the kidney. Applicant randomized mice with HIO engraftments at our 6-week time point following transplant to sham (transection and reanastomosis) or ICR groups and quantified morphometric factors associated with intestinal adaptation including crypt depth, villus height, epithelial proliferation, crypt fission and thickness of the circular and longitudinal smooth muscle layers (tunica muscularis) (FIG. 4A). Adaptation in NSG mice was confirmed with significant differences between sham and ICR groups for villus height, crypt fission (FIG. 4B-4D), crypt depth and smooth muscle layer thickness (FIG. 11A-11D), whereas there was no difference in proliferative index between sham and ICR groups (data not shown). In engrafted HIOs, ICR increased villus height, crypt fission (FIG. 4E-4G and FIG. 11E, 11F) and proliferative index (FIG. 4H, I) compared to sham-operated groups. These findings support the hypothesis that engrafted HIOs respond to humoral factors. In addition, this model may provide insights into the humoral adaptive response following intestinal resection. Functionally, engrafted HIOs expressed active brush border enzymes and exhibited a functional intestinal epithelial barrier, as shown by a permeability assay with FITC-dextran (FIG. 12A, 12B). The engrafted HIO epithelium was also capable of peptide uptake, reflecting the existence of absorptive functions (FIG. 12C).

To our knowledge, this is the first report of the development of a functional model for human small intestine in vivo derived from hPSCs. Our study highlights the potential of both ESCs and iPSCs to produce diverse cell types and tissue layers that mature following engraftment. Furthermore, the adaptive response seen in our human grafts following surgical resection in mouse hosts supports the role of humoral factors in this adaptive response and validates the use of our model for further in vivo studies of human small intestine. HIOs may also serve, through further translational research, as a means for the eventual treatment of short bowel syndrome and other gastrointestinal diseases, as they serve as a way to ‘personalize’ and bioengineer functional human intestine.

### Methods

### Animals

Immune-deficient NOD-SCID IL-2Rγnull (NSG) mice, 8-16 weeks old, were used in all experiments (obtained from the Comprehensive Mouse and Cancer Core Facility, Cincinnati, Ohio). All mice were housed in the animal facility at the Cincinnati Children's Hospital Medical Center (CCHMC). All experiments were performed with the approval of the Institutional Animal Care and Use Committee of CCHMC.

### Generation and Maintenance of Human Intestinal Organoids

Human intestinal organoids were generated and maintained as previously described (Spence et al, Nature 2011 and McCracken et al. Nat. Protoc. 2011). Human embryonic stem cells and induced pluripotent stem cells were grown in feeder-free conditions in six-well Nunclon surface plates (Nunc) coated with Matrigel (BD Biosciences) and maintained in mTESR1 media (Stem Cell Technologies). For induction of definitive endoderm (DE), human ES or iPS cells were passaged with Dispase or Accutase (Invitrogen) and plated at a density of 100,000 cells per well in a Matrigel-coated, Nunclon surface 24-well plate. For Accutase split cells, 10 μM Y27632 compound (Sigma) was added to the media for the first day. Cells were allowed to grow until they reached 80-95% confluence. Cells were then treated with 100 ng ml−1 of Activin A for 3 d as previously described (D′Amour et al. Nat. Biotechnol. 2005). DE was then treated with hindgut induction medium (RPMI 1640, 2 mM L-glutamine, 2% decomplemented FBS, penicillin-streptomycin and 100 ng ml−1 Activin A) for 4 d with 500 ng mL−1 FGF4 (R&D) and 3 μM Chiron 99021 (Tocris) to induce formation of mid-hindgut spheroids. Spheroids were then plated in Matrigel (BD) and maintained in intestinal growth medium (Advanced DMEM/F-12, N2, B27, 15 mM HEPES, 2 mM L-glutamine, penicillin-streptomycin) supplemented with 100 ng ml−1 EGF (R&D) and 100 ng ml−1 Noggin (R&D) to generate human intestinal organoids (HIOs). Media was changed at 3 d with Noggin removed and then changed twice weekly thereafter. HIOs were replated in fresh Matrigel every 14 d.

### Generation and Characterization of Induced Pluripotent Stem Cell Lines

For iPSC generation, fibroblasts were transduced with recombinant VSV-G-pseudotyped polycistronic lentiviral particles co-expressing reprogramming factors Oct4, Klf4, Sox2, cMyc and dTomato28. Nucleofected fibroblasts were then plated on hESC-qualified Matrigel, and 3-5 d post transduction, MEF media was replaced with mTeSR1, and cultures were subsequently fed daily with mTeSR1. After ˜3 weeks, putative iPSC colonies were identified and exposed to dispase for 5 min. Discrete colonies were manually excised and replated in mTeSR1 on Matrigel-coated dishes. Several lines with typical hESC-like morphology were then expanded independently in mTeSR1 on Matrigel-coated dishes. For passaging, iPSCs were exposed to dispase for 5 min, washed, and gently triturated before replating. iPSC lines were cryopreserved in mFreSR (StemCell Technologies). All cultures were maintained in a 5% CO2/air environment. All the experiments with iPSCs in this study were approved by institutional Embryonic Stem Cell Research Oversight (ESCRO).

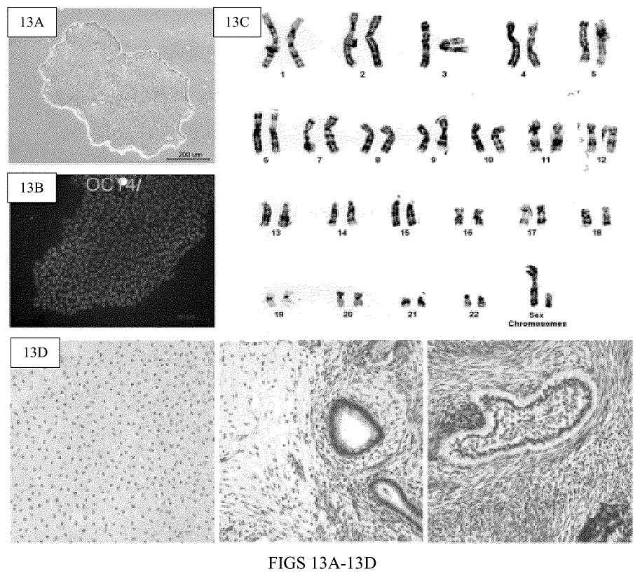

For analysis of pluripotency marker expression, iPSC cultures were fixed for 10 min at room temperature with 3.7% paraformaldehyde in PBS. Cells were then permeabilized for 10 min with PBS containing 0.5% Triton X-100 and incubated for 30 min at room temperature in blocking buffer (10% normal donkey serum in PBS). Antibodies to human Oct4 (Santa Cruz, sc-5279) and Nanog (Abcam, ab21624) were diluted in blocking buffer at 1:500 and incubated with cells overnight at 4° C. After incubation with fluorescent-labeled secondary antibodies, cultures were visualized using fluorescent microscopy. 4′,6-diamidino-2-phenylindole (DAPI) was used for nuclear counterstaining (FIG. 13A, 13B). Standard G-banded karyotype analyses of iPSCs was performed by the Cytogenetics Laboratory at Cincinnati Children's Hospital Medical Center. At least 20 metaphases were analyzed (FIG. 13C). Analysis of in vivo differentiation capacity was assessed with teratoma assays. Undifferentiated iPSCs were injected subcutaneously into immune compromised NOD/SCID GAMMA C^{-/-} mice. Teratomas formed within 6-12 weeks. Teratomas were excised, fixed, embedded in paraffin, sectioned and stained with H&E for histological examination (FIG. 13D).

### Generation of LGR5:eGFP BAC Transgenic Reporter hESC Line

The LGR5: eGFP bacterial artificial chromosome (BAC) transgenic reporter hESC line was generated. In summary, the BAC RP11-59F15 was obtained from the Children's Hospital Oakland Research Institute (http://bacpac.chori.org/) and grown in SW10535 cells. A single colony was expanded in LB+cam at 32° C., and recombineering proteins were induced by incubation at 42° C. for 20 min. The recombination cassette consisted of eGFP-FRT-PGKgb2-neo/kan-FRT, 50-bp homology region in LGR5, and a 20-bp homology region to peGFP-PGKneo. The homology regions were selected to replace the initiator methionine of LGR5 with that of eGFP followed by a bovine growth hormone polyadenylation signal and FRT-flanked bifunctional kanamycin/G418 resistance cassette. The recombination cassette was electroporated into SW105 cells, and cells were selected on plates with cam and kanamycin (kan; 50 μg ml−1). Clones were analyzed for properly targeted LGR5 BAC by PCR and confirmed by sequencing and nucleofected into single-cell suspensions of H9 hESCs using the Amaxa Human Stem Cell Nucleofector Starter Kit. Cells were selected in G418 (200 ng ml−1) for 2 weeks. G418-resistant cells were maintained in antibiotic indefinitely.

### Transplantation of Human Intestinal Organoids

NSG mice were kept on antibiotic chow (275 p.p.m. Sulfamethoxazole and 1,365 p.p.m. Trimethoprim; Test Diet). Food and water was provided ad libitum before and after surgeries. A single HIO, matured in vitro for 35 d, was removed from Matrigel, washed with cold phosphate-buffered saline (DPBS; Gibco), and embedded into purified type I collagen (rat tail collagen; BD Biosciences) 12 h before surgery to allow for formation of a solidified gel plug. These plugs were then placed into standard growth media overnight in intestinal growth medium (Advanced DMEM/F-12, B27, 15 mM HEPES, 2 mM L-glutamine, penicillin-streptomycin) supplemented with 100 ng ml−1 EGF (R&D). HIOs were then transplanted under the kidney capsule. Briefly, the mice were anesthetized with 2% inhaled isoflurane (Butler Schein), and the left side of the mouse was then prepped in sterile fashion with isopropyl alcohol and povidine-iodine. A small left-posterior subcostal incision was made to expose the kidney. A subcapsular pocket was created and the collagen-embedded HIO was then placed into the pocket. The kidney was then returned to the peritoneal cavity and the mice were given an IP flush of Zosyn (100 mg/kg; Pfizer Inc.). The skin was closed in a double layer and the mice were given a subcutaneous injection with Buprenex (0.05 mg/kg; Midwest Veterinary Supply). At 6 weeks following engraftment, the mice were then humanely euthanized or subjected to further experimentation.

### Intestinal Resections in Transplanted Mice

Male NSG mice that have previously been transplanted with HIOs 6 weeks prior were randomized to ileocecal resection (ICR) or sham operation. Mice were placed on liquid diet (Jevity 1Cal; Abbott) 24-48 h before surgery and were changed from antibiotic chow to liquid antibiotic (200 mg Sulfamethoxazole and 40 mg Trimethoprim oral suspension 5 mL−1; Hi-Tech Pharmacal) in their drinking water (0.3 mg mL−1 Trimethoprim) for the remainder of the experiment. Surgeries were completed under anesthesia as described above, and the abdomen of the mouse was opened anteriorly to expose the intestine. An average of 13.6 cm of distal-most small intestine was removed in addition to the cecum as previously described 26. Mice were kept in an incubator at 30° C. for 48 h after surgery and were then euthanized on post-operative day 7.

### Culture of Transplant-Derived Enteroids

Human enteroids were generated as previously described12,29. Briefly, engraftments were harvested and opened and pinned with mucosa facing upwards and then rinsed in cold PBS (Gibco). The tissues were then transferred to 2 mM EDTA (EDTA; Sigma-Aldrich) in PBS and rocked for 30 min on ice. After EDTA chelation, tissues were again washed in cold PBS, and crypts were manually removed from underlying submucosa and then filtered through a 100-μm cell strainer (Fisher Scientific). Crypts were then pelleted and resuspended in Matrigel (BD Biosciences) and overlaid with intestinal growth medium (Advanced DMEM/F-12, N2, B27, 15 mM HEPES, 2 mM L-glutamine, penicillin-streptomycin) supplemented with EGF (50 ng mL−1), Noggin (100 ng mL−1), R-spondin 1 (1 μg mL−1) (R&D Systems), 50% Wnt3a conditioned medium, 1 mM N-acetylcysteine, 10 nM (Leu15)-Gastrin, 10 mM Nicotidamide, 10 μM SB202190 (Sigma-Aldrich), 500 nM A-83-01 (Tocris). 2.5 μM Thiazovivin and 2.5 μM CHIR99021 (Stemgent) were added to the medium for the first 2 d. Medium was changed every 3 d. Established enteroids were passaged over time by enzymatic (TrypLE Express, Life Technologies) and mechanical dissociation through an 18-gauge needle after 7 d in culture. Dissociated enteroids were then resuspended in PBS and pelleted before resuspension in Matrigel and culture conditions as above.

### Intestinal Alkaline Phosphatase Activity

Frozen sections were washed in PBS and blocked using donkey serum. Sections were incubated with Permanent Red working solution (Permanent Red substrate+Permanent Red chromogen; Dako) for 30 min at room temperature. Sections were then washed in PBS and mounted for imaging.

### In Vivo FD4 Permeability Assay

FITC-conjugated dextran (FD4; 4,400 MW; Sigma) was dissolved in sterile water at a final concentration of 20 mg ml−1. Mice with previous transplants 8 weeks prior were then anesthetized as described above, and a left, posterior subcostal incision was made to expose the left kidney and engrafted intestinal tissue. Human engraftments were then injected each with 100 μL of FD4. Whole blood was then collected using heparinized hematocrit capillary tubes at time points 30 min and 4 h post-injection, and fluorescence intensity in murine sera was analyzed using a fluorescent plate reader. The concentration of FITC-dextran was then determined by comparison to the FITC-dextran standard curve dissolved in water.

### In Vivo Tomato Lectin Injections

Fluorescein Lycopersicon esculentum (tomato) lectin (Vector laboratories) was resuspended in PBS at a final concentration of 2 mg ml−1. 8 weeks after HIO transplantation, mice were placed under anesthesia as described above and were each injected with 200 μl of the tomato lectin via the tail vein. Mice were then humanely euthanized 30 min following injection, and tissues were collected for imaging.

### In Vivo D-Ala-Leu-Lys-AMCA Uptake Study

D-Ala-Leu-Lys-7-amido-4-methylcoumarin (D-Ala-Leu-Lys-AMCA; Sigma) was prepared as previously described 30 with modification for a final concentration of 25 μM solution in DMEM (Dulbecco's Modified Eagle Medium; Gibco). Mice were anesthetized as described above and a left posterior subcostal incision was created to expose the engraftment. The engraftment lumen was then injected with 100 μL of D-Ala-Leu-Lys-AMCA, and mice were closed in a double-layer fashion. Mice were euthanized 30 min post-injection, and tissue was collected for analysis. For comparison, engraftments were also injected with vehicle (DMEM solution) alone or with peptide solution mixed with 1 mM Captopril (Sigma), a competitive inhibitor of peptide uptake.

### Tissue Processing, Immunofluorescence and Microscopy

Tissues were fixed for 2 h to overnight in 4% paraformaldehyde (PFA). Organoid engraftments were frozen in OCT, whereas mouse intestinal tissues were embedded in paraffin. OCT sections were blocked using donkey serum (10% serum in 1×PBS plus 0.5% Triton-X) for 30 min and incubated with primary antibody overnight at 4° C. Slides were then washed and incubated in secondary antibody in blocking buffer for 2 h at room temperature (23° C.) Paraffin sections were deparaffinized, subjected to antigen retrieval, and stained in a similar fashion to OCT sections. Please see Table 1 for a list of antibodies and respective dilutions. Slides were then washed and mounted using ProLong Gold antifade reagent with DAPI (Life Technologies). Images were captured on a Nikon Eclipse Ti and analyzed using Nikon Elements Imaging Software (Nikon). For transmission electron microscopy (TEM), tissues were fixed overnight in 3% glutaraldehyde in 0.175 M sodium cacodylate buffer pH 7.4. Samples were then post fixed in 1% Osmium tetroxide in 0.175 M cacodylate buffer for 1 h at 4° C. Samples were washed and put through a series of graded ethanol (25, 50, 75, 95, 2× 100%). Infiltration was performed with 2× Propylene Oxide followed by graded infiltration with LX-112. Samples were placed in polymerization oven at 37° C. overnight and then kept at 60° C. for 3 d. A Hitachi H7600 transmission electron microscope was used to image TEM sections. For whole-mount staining, tissues were processed similarly as above and then cleared in Murray's solution. Imaging was performed with a Nikon A1 confocal microscope.

TABLE 1
List of primary and secondary antibodies used for immunostaining.

| | | | | | Cat. | |
|---|---|---|---|---|---|---|
| Provider | Label | Host | Reactivity | Name | Number | Dilution |
| Primary Ab | | | | | | |
| Abcam | | Goat | Mouse, | Vimentin (VIM) | ab11256 | 1:500 |
| | | | Human | | | |
| Sigma | | Mouse | Human | aplha SMA (α-SMA) | Ab8207 | Prediluted |
| R&D | | Goat | Mouse, | E-cadherin (Ecad) | AF648 | 1:500 |
| | | | Human | | | |
| R&D | | Rat | Mouse, | E-cadherin (Ecad) | MAB 7481 | 1:500 |
| | | | Human | | | |
| Dako | | Rabbit | Human | Lysozyme (Lyz) | A0099 | 1:1000 |
| Immunostar | | Rabbit | Mouse, | Chromogranin | 20085 | 1:500 |
| | | | Human | A (ChgA) | | |
| Santa-Cruz | | Rabbit | Mouse, | Mucin 2 (MUC2) | sc-15334 | 1:200 |
| | | | Human | | | |
| Millipore | Cy 3 | Mouse | Human | Human Nuclear | MAB | 1:500 |
| | | | | (HuNuc) | 1281C3 | |
| Biogenex | | Mouse | Human | CDX2-88 (CDX2) | AM392 | 1:300 |
| Sigma | | Rabbit | Human | Lactase- | HPA007408 | 1:500 |
| | | | | gycosylceramidase | | |
| | | | | (LCT) | | |
| Novus | | Rat | Mouse | MECA-32 | NB100- | 1:500 |
| Biologicals | | | | | | |
| Santa-Cruz | | Goat | Mouse, | Gastric Inhibitory | 77668 | 1:500 |
| | | | Human | Peptide (GIP) | | |
| Santa-Cruz | | Goat | Mouse, | Villin | sc-23554 | 1:100 |
| | | | Human | | | |
| R&D | | Goat | Mouse, | DPPIV | sc-7672 | 1:500 |
| | | | Human | | | |
| DSHB | | Mouse | Human | Sucrase- | AF954 | 1:500 |
| | | | | Isomaltase (SIM) | | |
| eBioscience | eF 660 | Rat | Mouse | Lyve-1 | HBB2/614/88 | 1:500 |
| Santa-Cruz | | Rabbit | Mouse, | Peptide | 50-0443 | 1:500 |
| | | | Human, | Transporter 1 | | |
| | | | Rat | (Pept1) | | |
| Millipore | FITC | Mouse | Human | Achaete Scute | Sc-20653 | 1:20 |
| | | | | homolog 2 | | |
| | | | | (ASCL2) | | |
| Pierce | | Mouse | Human | CD31/PECAM-1 | FCMAB285F | 1:500 |
| Thermo | | | | | | |
| Secondary | | | | | MA5-15336 | |
| Ab | | | | | | |
| Invitrogen | AF488 | Donkey | Goat | Goat IgG | | 1:500 |
| Invitrogen | AF555 | Goat | Rabbit | Rabbit IgG | A11055 | 1:500 |
| Invitrogen | AF488 | Donkey | Rat | Rat IgG | A21428 | 1:500 |
| Invitrogen | AF488 | Donkey | Rabbit | Rabbit IgG | A21208 | 1:500 |
| Invitrogen | AF546 | Donkey | Mouse | Mouse IgG | A21206 | 1:500 |
| Invitrogen | AF647 | Donkey | Goat | Goat IgG | A10036 | 1:500 |
| Invitrogen | AF555 | Goat | Mouse | Mouse IgG2a | A21447 | 1:500 |
| Invitrogen | AF546 | Donkey | Rabbit | Rabbit IgG | A21137 | 1:500 |
| Invitrogen | AF555 | Donkey | Goat | Goat IgG | A10040 | 1:500 |
| Invitrogen | AF488 | Rabbit | Goat | Goat IgG | A21432 | 1:500 |
| Invitrogen | AF488 | Donkey | Mouse | Mouse IgG | A11078 | 1:500 |
| Invitrogen | AF568 | Donkey | Mouse | Mouse IgG | A21202 | 1:500 |
| Jackson-Imm. | Cy5 | Goat | Rat | Rat IgG | A10037 | 1:500 |

### RNA Isolation and RT-qPCR

RNA was extracted using RNeasy Plus Mini Kit (Qiagen) according to manufacturer's protocols. A cDNA reverse transcription kit (Applied Biosystems) was used to synthesize cDNA. Taqman (Applied Biosystems) gene expression assays were then performed on triplicate samples using a OneStep cycler (Applied Biosystems). See Table 2 for a list of Taqman probes used.

TABLE 2
List of Human Taqman ® Probes used for RT-PCR.

| Human | | | | | Amplicon | |
|---|---|---|---|---|---|---|
| TaqMan ® Probes | | Gene | | | Length | |
| Gene | Gene Name | ID | TaqMAN # | Accession # | (bp) | Maker type |
| ALPI | Alkaline | 248 | Hs00357579_g1 | NM_001631.3 | 56 | Enterocyte |
| | Phosphatase, | | | | | |
| | intestinal | | | | | |
| CHGA | Chromogranin | 1113 | Hs00900375_m1 | NM_001275.3 | 88 | Enteroendocrine |
| | A | | | | | cell |
| LYZ | Lysozyme | 4069 | Hs00426232_m1 | NM_000239.2 | 67 | Paneth cell |
| MUC2 | Mucin 2 | 4583 | Hs03005103_g1 | NM_002457.2 | 53 | Goblet cell |
| EpCAM | Epithelial Cell | 4072 | Hs00901885_m1 | NM_002354.2 | 95 | Epithelium/ |
| | Adhesion | | | | | Enterocyte |
| | Molecule | | | | | |
| VIM | Vimentin | 7431 | Hs00185584_m1 | NM_003380.3 | 73 | Mesenchyme/ |
| | | | | | | Fibroblast |
| ACTA2 | alpha-2, | 59 | Hs00426835_g1 | NM_001141945.1 | 105 | Smooth |
| | Smooth | | | | | Muscle |
| | muscle actin | | | | | |
| DES | Desmin | 1674 | Hs00157258_m1 | NM_001927.3 | 83 | Fibroblasts/ |
| | | | | | | Smooth |
| SI | Sucrase- | 6467 | Hs00356112_m1 | NM_001041.3 | 64 | Enterocyte |
| | Isomaltase | | | | | |
| | (alpha- | | | | | |
| | glucosidase) | | | | | |
| VILL | Villin-like | 50853 | Hs00210626_m1 | NM_015873.3 | 74 | Enterocyte |
| DPP4 | Dipeptidyl- | 1803 | Hs00175210_m1 | NM_001935.3 | 90 | Enterocyte |
| | peptidase 4 | | | | | |
| SLC2A2 | Facilitated | 6514 | Hs01096908_m1 | NM_000340.1 | 65 | Enterocyte |
| | Glucose | | | | | |
| | Transporter 2 | | | | | |
| | (GLUT 2) | | | | | |
| GIP | Gastric | 2695 | Hs00175030_m1 | NM_004123.2 | 78 | Proximal |
| | Inhibitory | | | | | |
| | Polypeptide | | | | | |
| LCT | Lactase | 3938 | Hs00158722_m1 | NM_002299.2 | 79 | Enterocyte |

### Morphometric and Statistical Analyses

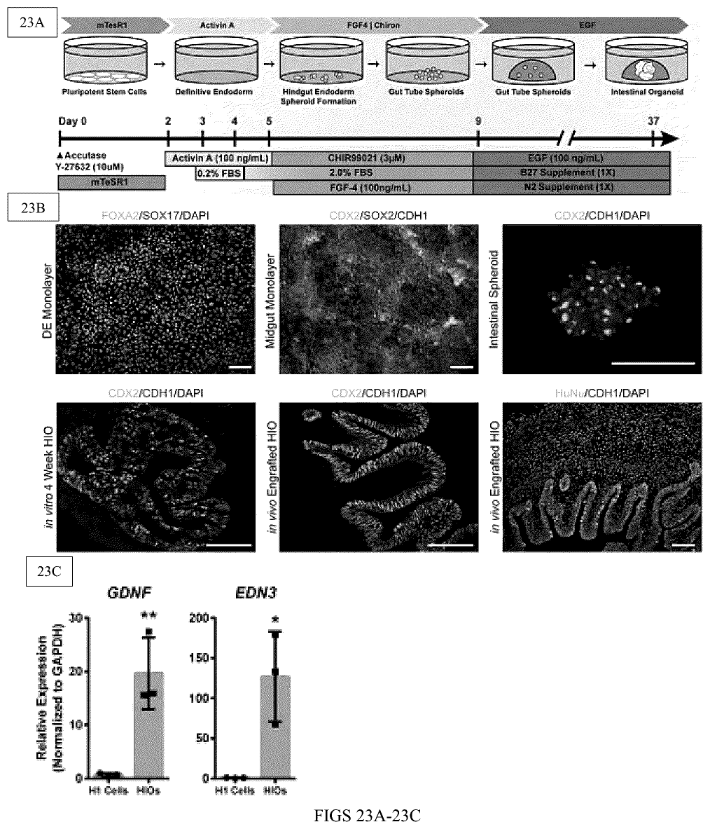

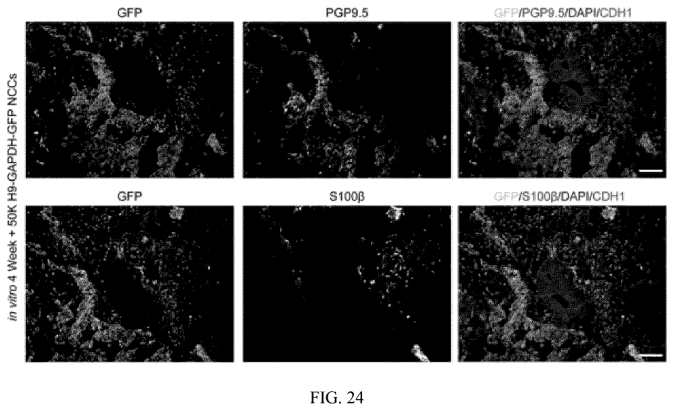

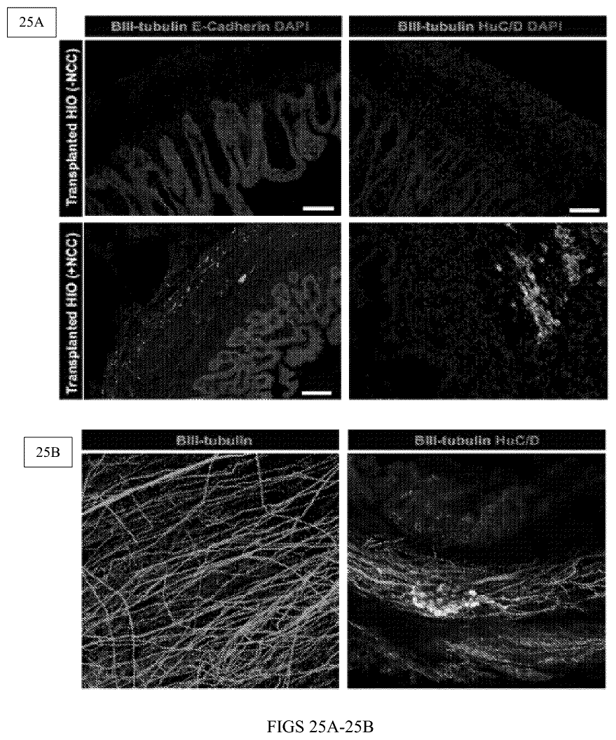

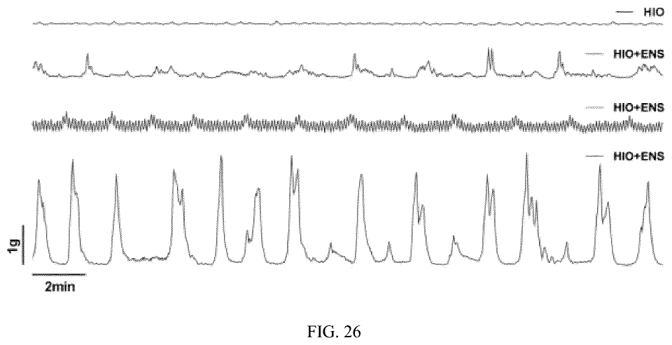

Histological sections were stained with hematoxylin and eosin or subjected to immunohistochemistry (as described above). All histological samples were counted in a blinded manner. Crypt depth, villus height, and smooth muscle layer (tunica muscularis) thickness were measured for a minimum of 100 well-oriented crypt-villus units or smooth muscle layer segments per mouse and then averaged using Nikon NIS imaging software. Crypt fission was also calculated in a similar manner using longitudinal sections to determine the percentage of fissioning crypts from at least 100 intact crypts per animal. A fissioning crypt is defined as a bifurcating crypt with a bisecting fissure creating two (or sometimes more) flask-shaped bases with a shared single crypt-villus junction (FIG. 23A, F). Proliferative index was determined by injecting mice with 5-ethynyl-2′-deoxyuridine (Edu) 2 h before killing, and then, following routine Edu immunohistochemistry according to manufacturer's instructions, proliferative index was determined by calculating the ratio of Edu-positive cells to total cells within at least 10 intact crypts. All data are represented as mean±s.e.m. t-tests and two-way analysis of variance were completed using Prism (GraphPad). The determined significance cutoff was P<0.05. No statistical method was used to predetermine sample size.

The experiments were not randomized except ICR experiments, where transplanted animals were randomly assigned to a sham or an ICR group. The investigators were not blinded to allocation during experiments and outcome assessment except for the morphometric analysis for the ICR experiments.

### Development of Tissues With a Functional Enteric System

### Generation of Vagal Neural Crest Cells

Cranial and trunk (both vagal and sacral) NCCs emanate from discrete regions along the A-P axis, and colonize different regions of the developing embryo. The ENS, for example, derives from vagal NCCs. Therefore, to generate human intestinal organoids with an ENS, Applicant hypothesized that vagal NCCs would be a more appropriate starting source. Given that numerous methods have been reported to direct the differentiation of PSCs into cranial NCCs 9,10, Applicant first started by generating cranial NCCs in step-wise manner that mimics normal NCC development (FIG. 1, FIG. 20). As previously reported, cranial NCCs derived from PSCs expressed stage-specific molecular markers in a manner similar to cranial NCCs during embryonic development including Sox2, Pax3, and Pax7 at early neuroepithelial stages and HNK-1, Sox10, Snail2 and vimentin in migrating neural crest cells.

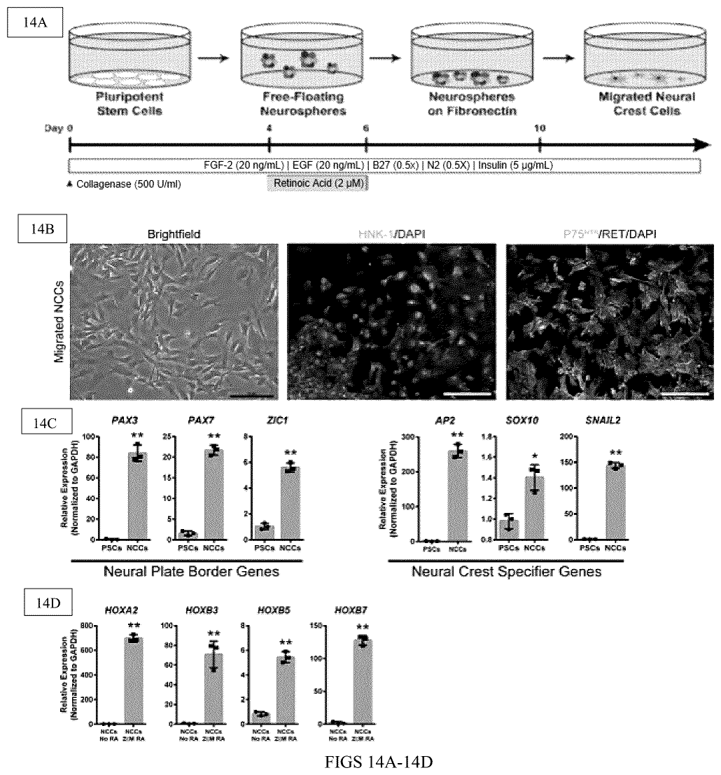

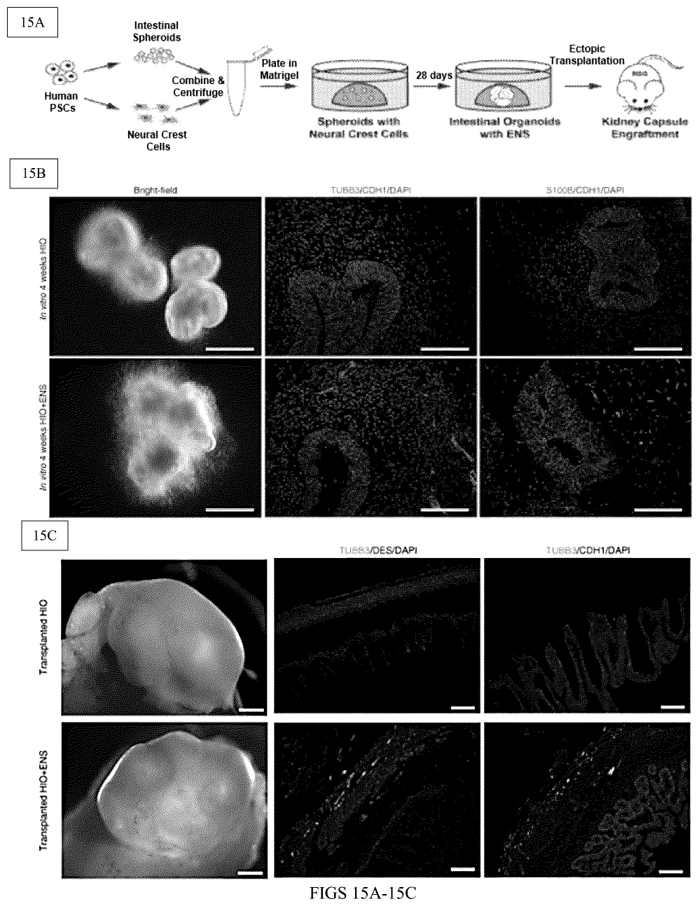

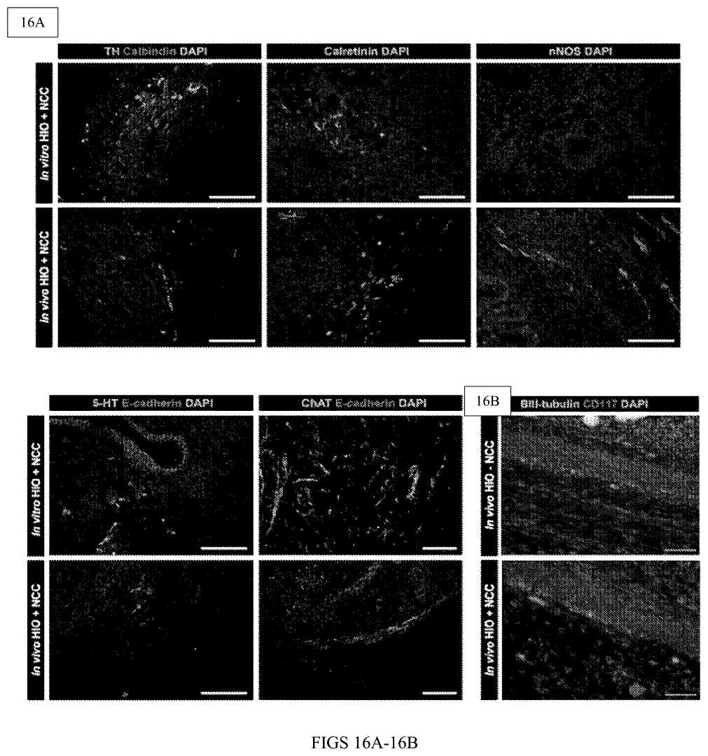

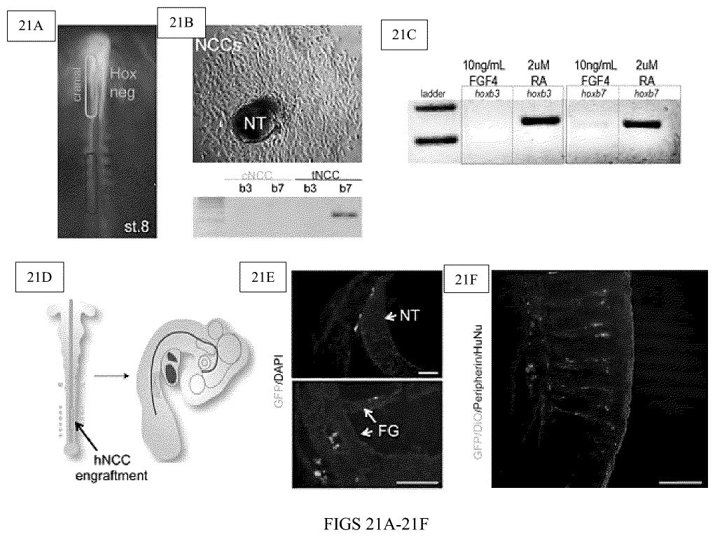

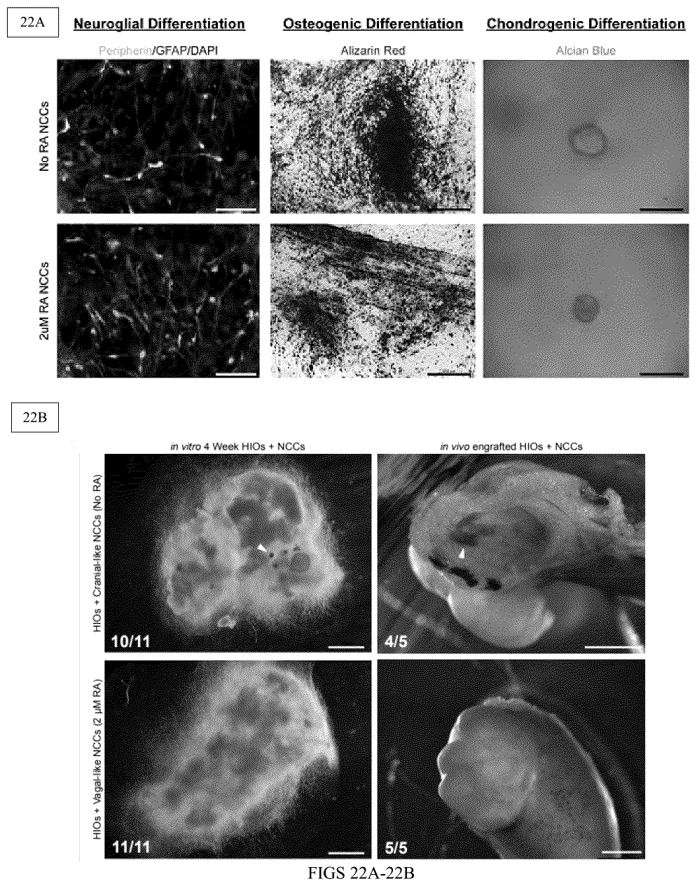

To generate vagal NCCs, which come from more posterior regions of the neural tube, Applicant manipulated signaling pathways that are known to promote a posterior fate at early stages during the differentiation process. Applicant found that retinoic acid (RA), but not FGF4 (data not shown), for two days at the neurosphere stage resulted in the robust expression of the vagal-level Hox genes HOXB3, HOXB5, and HOXB7 (FIG. 14D), similar to the observations by Mica et al. Applicant confirmed that the posterior Hox gene hoxb7 was expressed in trunk/vagal NCCs isolated from chick embryos (FIG. 21A and 21B). Moreover, RA was able to induce vagal Hox gene expression in chick cranial NCCs (FIG. 21C), consistent with the known role of RA in patterning of vagal NCCs during vertebrate development.

### Differentiation Potential of Neural Crest Cells

Multipotent NCCs can form ectoderm derivatives including neurons, glia, and melanocytes as well as cranial mesodermal lineages including osteocytes and chondrocytes. To test whether NCCs retained multipotent differentiation potential, Applicant directed differentiations of NCCs towards various ectomesenchymal lineages in vitro (FIG. 22A). NCCs were able to differentiate into Peripherin+ neurons and GFAP+ glial cells in vitro upon growth factor withdraw. PSC-derived NCCs could also be differentiated into mesoderm lineages including osteocytes and chondrocytes as indicated by positive staining for alizarin red and alcian blue, respectively. Applicant did not observe any gross differences of RA treatment on the in vitro differentiation potential of NCCs suggesting that the mulitpotency of PSC-derived cranial and vagal/Hox-positive NCC populations had been maintained. Applicant further tested if PSC-derived NCCs had neural crest cell behaviors in an embryonic context by injecting them into chick embryos. PSC-derived NCCs followed a migratory path similar to endogenous NCCs and differentiated into neurons (FIG. 21D-21F).

### Generating Human Intestinal Organoids (HIOs) With NCC-Derived Neuroglial Cells in Vitro

Applicant previously developed a method to differentiate human PSCs into intestinal organoids following a stepwise differentiation first into definitive endoderm, then into mid/hindgut tube spheroids that are expanded into 3D intestinal tissue (Spence et al., McCracken et al. (FIG. 23). In vitro, HIOs contain all major intestinal epithelial cells types, as well as a stratified mesenchyme containing subepithelial myofibroblasts and smooth muscle cells. When HIOs are engrafted into the kidney subcapsular space they continue to develop and form highly differentiated intestinal tissue with crypts and villi, functional intestinal stem cells, and layers of smooth muscle (FIG. 23B).

While HIOs contained most of the epithelial and mesenchymal cell types found in the developing gut, they did not contain an ENS. To incorporate NCCs/ENS precursors into developing HIOs, Applicant mechanically aggregated mid/hindgut spheroids with PSC-derived NCCs by low speed centrifugation and transferred aggregates to 3 dimensional growth conditions for 28 days (FIG. 15A). HIOs with and without NCCs were comparable in size (1-2 mm in diameter), however Applicant detected an abundance of βIII-tubulin (TUJ1)+ neurons and S100+ glia embedded within the mesenchyme of HIOs combined with NCCs (FIG. 15B). Applicant rarely detected neurons and never glia in HIOs without NCCs in vitro. Moreover Applicant confirmed the NCC origin of neuroglial cells by using NCCs that were derived from a PSC line that constitutively expressed GFP (FIG. 24). The spatial relationship between the epithelium, mesenchyme and neuroglial cells in HIOs+NCCs closely resembled that of human fetal intestine and E11.5 mouse small intestine (data not shown), suggesting that intrinsic cues in HIOs help organize the developing ENS cells. Consistent with this, HIOs expressed both GDNF and EDN3, two critical chemoattractants known to regulate NCC migration into the gut mesenchyme (FIG. 23C), suggesting that incorporation of PSC-derived NCCs into HIO mesenchyme may have utilized normal developmental pathways.

### Maturation of the ENS Following in Vivo Growth

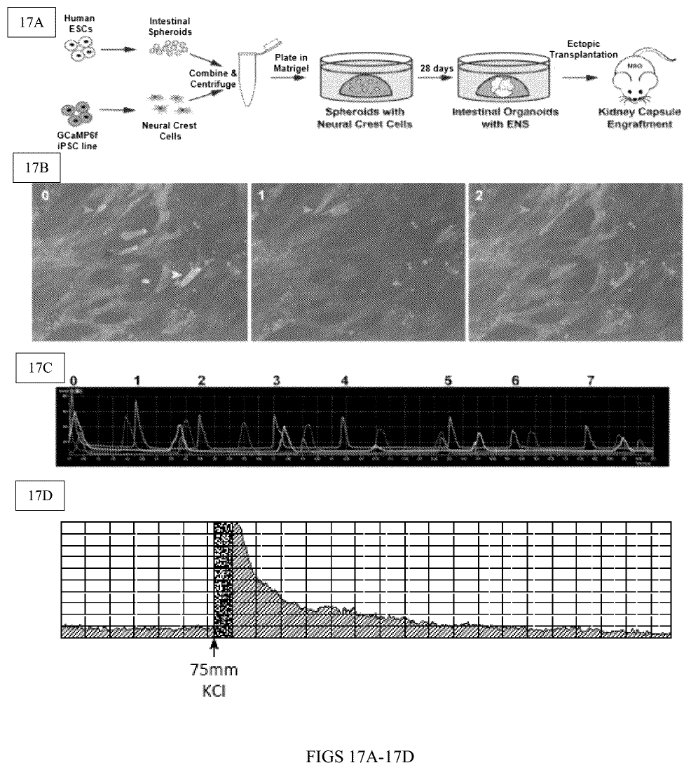

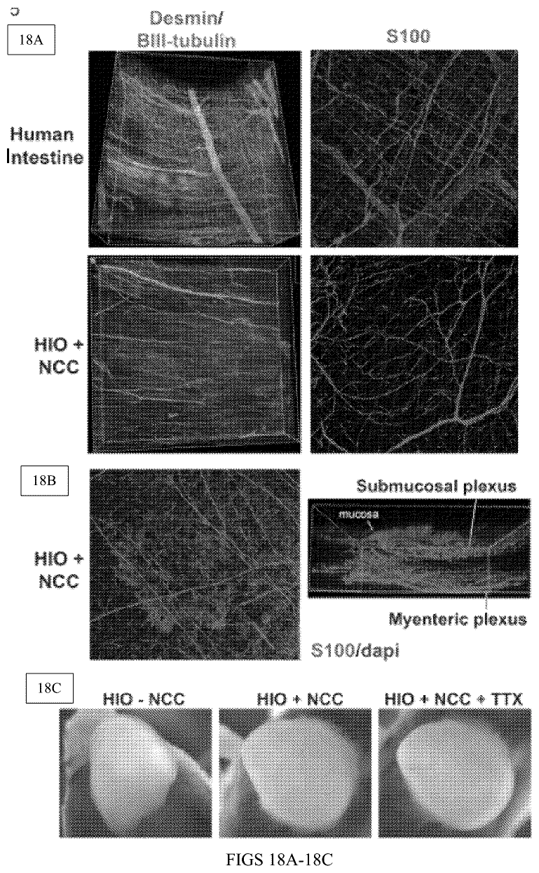

Our previous studies have demonstrated that HIOs that are engrafted into mice and allowed to grow for 6-10 weeks in vivo become vascularized, grow to 1-3 cm in diameter, and form highly mature intestinal tissues with villi and crypts containing functional intestinal stem cells as described above and in Watson et al 2011 (FIG. 17B). Moreover the HIO mesenchyme matures into submucosal and myenteric layers of smooth muscle fibers (FIG. 15C). Analysis of transplanted HIOs+PSC-derived NCCs revealed that neurons (βIII-tubulin) and glia (S100) were organized into multiple layers in proximity to the submucosal and myenteric layers of smooth muscle (FIG. 15C). Applicant did not detect neurons or glia in transplanted HIOs without NCCs. While these experiments were performed with vagal/HOX-positive NCC populations, PSC-derived cranial/HOX-negative NCCs were also competent to engraft and form ENS neuroglial cells. However cranial NCCs also formed pigmented epithelial cells (FIG. 22B) and cartilage (not shown) consistent with them having a broader multipotency.

### Neuronal Diversity and Function in HIOs+NCCs

The ENS is a complex network of excitatory and inhibitory neuronal subtypes, as well as interneurons. Examination of neural markers in HIOs+NCCs cultured in vitro for four weeks suggested a significant degree of neuronal diversity. Applicant observed tyrosine hydroxylase (TH), calbindin, calretinin, and serotonin (5-HT) positive cells, which are expressed by dopaminergic, excitatory and interneurons (FIG. 3A). However Applicant did not detect nNOS positive neurons in vitro. In contrast, in vivo transplanted HIOs+NCCs had an abundance nNOS+ neurons suggesting that the ENS matured during the period of in vivo growth. Many neurons appear to be associated with smooth muscle in transplanted HIOs+NCCs. Moreover, ENS neurons were associated with cells expressing CD117, a marker of interstitial cells of Cajal (ICCs) (FIG. 16B). While ICCs were present in HIOs lacking an ENS, there appeared to be fewer clusters of CD117-expressing cells in HIO−NCCs (data not shown), suggesting a developmental interaction between ENS cells and ICCs.

The accessibility of in vitro grown HIOs allowed us to test the functionality of neurons using live imaging with the Ca2+ sensor GCaMP6f19. To do this, Applicant generated HIOs and incorporated into them NCCs that were derived from a PSC line containing GCaMP6 (FIG. 17A). Imaging of neural activity in HIOs grown for 8 weeks in vitro detected broad, spontaneous, and rapid calcium transients within neurons. When individual neurons were monitored there was an obvious periodicity of calcium efflux in the range of 3-4 seconds/neuron (FIGS. 17B and 17C). To test if neurons were capable of undergoing coordinated activity Applicant exposed HIOs+GCaMP6 expressing NCCs to KCl (30 mM) and observed a large wave of neuronal calcium efflux (FIG. 17D).

### Neuroglial Plexus Formation in HIO+NCCs Grown in Vivo

Histological analysis of ENS neurons and glia suggested that they were forming a neural plexus within the smooth muscle layers of the HIOs. However this conclusion is difficult given the 2-dimensional nature of sectioned tissues. Applicant therefore performed 3 dimensional imaging of tissue from an HIOs+NCC matured in vivo via whole mount immunofluorescence using neuronal, glial and smooth muscle markers and compared these to human small intestine (FIG. 18). The ENS of human small bowel contained a network of both neurons (BIII-tubulin) and glia (S100) that integrated into smooth muscle layers (desmin+ cells). The ENS that formed within the developing HIO similarly contained a complex neural plexus of neurons and glia that appeared oriented with smooth muscle fibers (FIG. 18A). A lateral view of 3D images of HIO+NCC samples clearly showed evidence of a myenteric plexus (FIG. 18B) as well as a plexus that is located in the submucosa adjacent to the epithelium (blue—dapi). Applicant also observed clusters of neuronal cell bodies, both in sections and 3D imaging (FIG. 25), reminiscent of ganglia. Relative to human intestine, the ganglia in HIOs were smaller and less developed.

### ENS-Mediated Contractile Activity

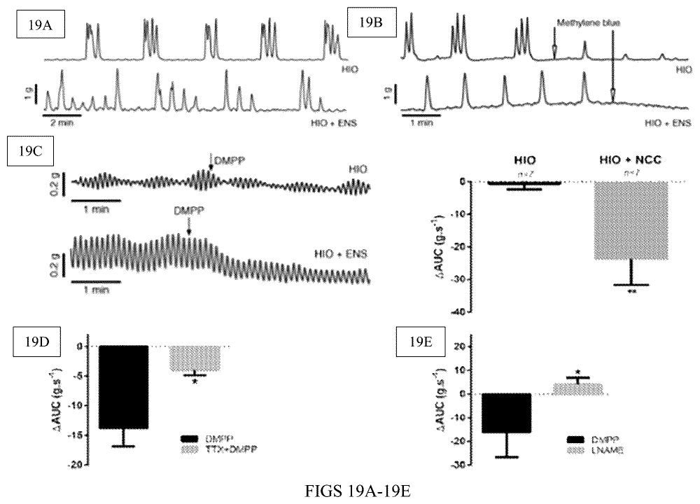

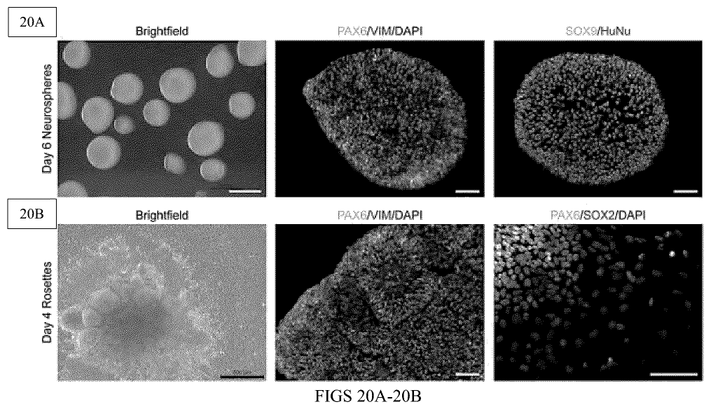

While the organization of epithelium, smooth muscle, neurons and glia in HIO+ENS tissues were similar to that of human intestine, it was not clear if there existed any neuromuscular communication in HIO+ENS tissues. To determine if the PSC-derived ENS could function in HIO tissues Applicant explanted kidney grafts into Tyrode's solution and subjected them to an electrical field stimulation (FIG. 19C). HIO tissue without an ENS subjected to a single 1 ms pulse at 100V (high voltage) caused a single contraction, suggesting the direct stimulation of smooth muscle contraction. In contrast, HIO tissue containing an ENS subjected to a single 1 ms pulse at 50V (low voltage) triggered a sustained wave of contractions. To determine if contractions were due to the activity of the ENS Applicant blocked neuronal activity with tetrodotoxin (TTX), which binds to voltage-gated Na+ channels on nerves, thus inhibiting the firing of action potentials. TTX completely inhibited the ability of low voltage stimulation to trigger motility in HIO+NCC tissue.

### Neuromuscular Coupling in HIO+NCC Tissues

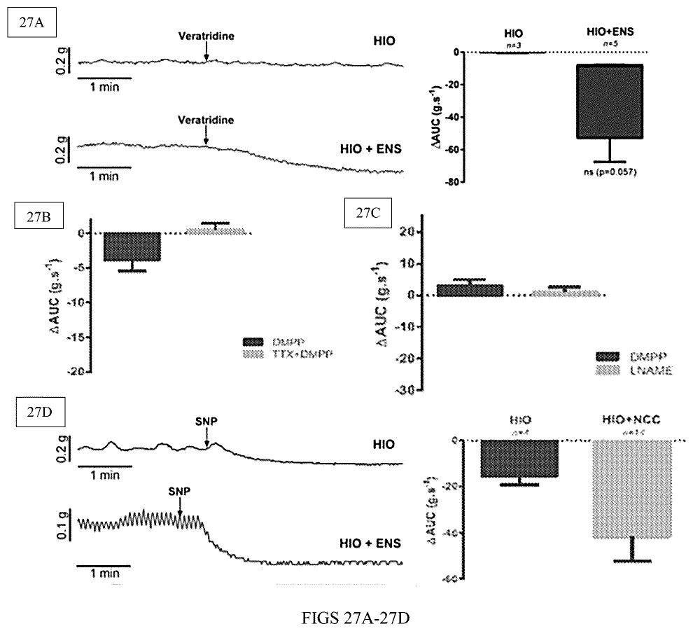

GI motility involves the coordination of ENS-dependent and independent contraction and relaxation of smooth muscle. To better dissect these processes Applicant isolated tissue strips from HIOs and HIO+NCCs and analyzed them using organ chamber experiments. In the absence of neuronal stimulation, Applicant observed spontaneous phasic contractions in both HIO and HIO+NCC tissues (FIG. 19A and FIG. 26A) consistent with the presence of ICCs in both conditions (FIG. 16B). Methylene Blue, which inhibits ICC activity, abolished phasic contractions (FIG. 19B). To identify ENS-dependent roles in contraction and relaxation Applicant activated neurons using a selective α3 nicotinic receptor agonist Dimethylphenylpiperazinium (DMPP) and veratridine, which increases nerve excitability by inhibiting Na+-channels inactivation. Pharmacological activation of the ENS induced a relaxation of the muscle in HIO+NCC tissues but not in HIOs alone (FIG. 6C and FIG. 25A). Applicant confirmed that relaxation was neuron-dependent using TTX, which inhibited the ability of the neurons to induce muscle relaxation in response to DMPP and or veratridine (FIG. 19D and FIG. 27B).

Applicant aimed at identifying a putative neuromediator responsible for the smooth muscle relaxation that was induced by the ENS. As Nitric Oxide (NO) is a well-known enteric inhibitory neuromediator and as nNOS-expressing neurons are abundant in transplanted in HIO+NCC tissues (FIG. 16) Applicant inhibited NO synthesis with NG-nitro-L-arginine methyl ester (L-NAME). L-NAME pretreatment significantly decreased the relaxation after DMPP stimulation thus indicating that NO was produced by the neurons to induce a relaxation (FIG. 19E and FIG. 27C). Applicant further stimulated NO release using Sodium Nitroprusside (SNP), and observed a relaxation of smooth muscle in HIO+NCC tissues (FIG. 27D).

### Methods

### Cell Lines and Culture Conditions

Human ES and iPS cells, H1 (WA-01), H9 (WA-09) and WTC11 AAVS1-CAG-GCaMP6f, were maintained in an undifferentiated state on Matrigel (BD Biosciences) without feeders. H9-GAPDH-GFP hESCs were generated by targeting sequences encoding GFP to the 3′ UTR of the GAPDH gene using standard procedures 33 that incorporated TALEN facilitated homologous recombination 34. GFP is expressed as T2A 35 pseudofusion protein immediately adjoining the c-terminus of GAPDH.

Cells were fed mTeSR1 media and routinely passaged using Dispase II (Gibco). NCCs and HIOs were generated and combined at an early stage of intestinal differentiation to generate HIOs containing ENS. Briefly, for NCC generation (FIG. 1), hPSCs were treated with collagenase IV (500 U/mL) in mTeSR1 for 60 min to detach colonies. Cells were washed to remove collagenase, then gently triturated and resuspended in neural induction media on non-TC treated petri dishes. Neural induction media was changed daily and retinoic acid (2 μM) was added on days 4 and 5 for posteriorization. Day 6 free-floating neurospheres were plated on fibronectin (3 μg/cm2) and fed neural induction media without RA for 4 days. Migrated cells were collected using a brief Accutase treatment (2-3 min) and passaged onto fibronectin or used immediately for combining with HIOs. NCCs were differentiated essentially as described for neuroglial 9 and mesenchymal lineages 36. Applicant generated HIOs in a similar manner to our previous protocols similar to that described above, but most notably used small molecule CHIR99021 in place of WNT3A (FIG. 22). Following the formation of gut tube spheroids, Applicant gently centrifuged spheroids+/−NCCs and embedded them in Matrigel. Cultures were fed a basic gut media (advanced DMEM/F12, 1×B27 supplement, 1×N2 supplement, 10 μM HEPES, 2 mM L-glutamine, 1×Pen-Strep) supplemented with 100 ng EGF mL−1 and maintained in vitro for up to 8 weeks.

### In Vivo Transplantation and Electrical Field Stimulation of HIOs

HIOs+/−NCCs were ectopically transplanted into the kidney capsule of NOD/SCID-gamma (NSG) mice following the protocol described above and in Watson et al. Briefly, 4-6 week HIOs were embedded in collagen and transplanted into the kidney subcapsular space. Engrafted HIOs were harvested 6-10 weeks following transplantation and analyzed for neural and glial markers or used for electrical field stimulation (EFS) experiments. For EFS, HIOs were explanted into Tyrode's solution and equilibrated for approximately 5 min before beginning stimulation. Electric stimulation was applied using a Grass S88 Stimulator (Grass Technologies) with single pulse, 1 ms duration, and 50 or 100 V settings. HIOs were then incubated for 5 min in 10 μM TTX diluted in Tyrode's, rinsed, and placed back in fresh Tyrode's. EFS was then repeated. Movies were recorded on a Leica dissection microscope using Leica Application Suite software and processed with VideoLAN and Handbrake to achieve 16× play speed.

### Chick Embryo Manipulation

Chicken eggs were purchased from Charles River and incubated at 39° C. until they reached the desired Hamburger and Hamilton stage. For chicken NCC culture, dorsal neural tubes from cranial or trunk region of HH8 embryos were dissected by Gastromaster and cultured on matrigel-coated tissue culture dish with NCC culture medium (BajPai R et al, 2010) for 24 hours. The neural ectoderm was scrapped off the plate by tungsten needle after NCCs migrated out from the neural tube explants. The NCCs remained on the plate were then treated with 4 ng/ml FGF4 or 2 μM Retinoic acid for 48 hours. For NCCs injection, GFP-labeled human PSC-derived NCCs were collected and injected intersomitically into HH10-12 chicken embryos. The embryos were harvested around HH38 for analysis.

### Immunohistochemistry and Microscopy

NCCs, cell monolayers, and day 0 spheroids were fixed with 4% paraformaldehyde (PFA) at 23° C. for 15 min, washed and then stained directly. Four week in vitro HIOs and in vivo transplants were fixed in 4% PFA at 4° C. for 1 hour to overnight. Tissues were processed and embedded in OCT, sectioned at approximately 10 μm, and affixed to Superfrost Plus slides (Fisherbrand). Frozen sections and cells were permeabilized with 0.25% Triton-X100 for 10 min then blocked with 5% Normal Donkey Serum (NDS, Jackson Immunoresearch) in PBS for 30 min at 23° C. Primary antibodies diluted in PBS were applied to slides and cells overnight at 4° C. followed by washes and incubation with secondary antibodies at 23° C. for 2 hours (See Table 3 for antibodies and dilutions). Slides were mounted using Fluromount-G and images were obtained using a Zeiss ApoTome Imager Z1 or Zeiss LSM510 confocal microscope. Whole mount 3D images were obtained by fixing tissues in 4% PFA overnight at 4° C. then equilibrating in 100% methanol for 1 hour on ice. Tissues were permeabilized with Dent's bleach (4:1:1 MeOH:DMSO:30% H2O2) for 2 hours at room temp before rehydrating with PBS. Tissues were then blocked with 5% NDS in PBS for 2 hours at room temp, incubated with primary antibodies in PBS overnight at 4° C., washed, and incubated with secondary antibodies in PBS overnight at 4° C. Following staining, tissues were dehydrated through a methanol series and cleared with Murray's Clear (2:1 Benzyl benzoate:Benzyl alcohol) prior to imaging on a Nikon A1 inverted confocal microscope. Images were processed using NIS Elements, Bitplane Imaris, Zeiss Axiovision, and Adobe Photoshop CS7 softwares.

TABLE 3

| | Source | Dilution |
|---|---|---|
| Primary Antibody | | |
| Goat anti-E-Cadherin | R&D #AF648 | 1:250 |
| Rat anti-E-Cadherin | R&D #MAB7481 | 1:1000 |
| Rabbit anti-BIII-tubulin (TUJ1) | Abcam #ab18207 | 1:2000 |
| Chicken anti-BIII-tubulin (TUJ1) | Abcam #ab41489 | 1:1000 |
| Rabbit anti-c-kit/CD117 (YR145) | Abcam #ab32363 | 1:200 |
| Mouse anti-HuC/D | Invitrogen #A21271 | 1:40 |
| Rabbit anti-PGP9.5 | Dako #Z511601 | 1:1000 |
| Mouse anti-HNK-1 | DSHB #1C10 | 1:50 |
| Mouse anti-p75^{NTR} | ATS #AB-N07 | 1:200 |
| Rabbit anti-Ret | Aviva #ARP30878 | 1:200 |
| Goat anti-Peripherin | Santa Cruz #sc-7604 | 1:200 |
| Mouse anti-GFAP | Millipore #MAB360 | 1:500 |
| Rabbit anti-S100 | Dako #Z031129 | 1:1000 |
| Rabbit anti-nNOS | Abcam #ab76067 | 1:1000 |
| Goat anti-Desmin | Santa Cruz #sc-7559 | 1:100 |
| Goat anti-Calretinin | Millipore #Ab1550 | 1:2000 |
| Rabbit anti-Calbindin | Gift from K. Campbell | 1:4000 |
| Chicken anti-TH | Aves #TYH | 1:500 |
| Mouse anti-human Nuclei | Chemicon #MAB1281 | 1:200 |
| Secondary Antibody | | |
| Donkey anti-mouse 488 | Jackson Immuno | 1:500 |
| Donkey anti-mouse 546 | Invitrogen | 1:500 |
| Donkey anti-goat 568 | Invitrogen | 1:500 |
| Donkey anti-rabbit 546 | Invitrogen | 1:500 |
| Donkey anti-rabbit 647 | Jackson Immuno | 1:500 |
| Donkey anti-chicken 549 | Invitrogen | 1:500 |

### RNA Isolation and Quantitative Real-Time PCR

Total RNA from cells and organoids was isolated using NucleoSpin RNA isolation Kit (Macherey-Nagel). Complementary DNA was generated immediately following RNA isolation using SuperScript VILO cDNA Synthesis Kit (Invitrogen). Both isolation and synthesis were carried out according to the manufacturers' protocols. Applicant performed qPCR using QuantiTect SYBR Green PCR Kit (Qiagen) on a BioRad CFX96 Real-Time PCR Detection System. Primer sequences were generally obtained from qPrimerDepot (http://primerdepot.nci.nih.gov) and can be found in Table 4.

TABLE 4

| Gene | Orientation | Primer Sequence |
|---|---|---|
| AP2α | Sense | ATGCTTTGGAAATTGACGGA |
| | Anti-sense | ATTGACCTACAGTGCCCAGC |
| Edn3 | Sense | GCACGTGCTTCACCTACAAG |
| | Anti-sense | GGACAGTCCATAGGGCACC |
| GAPDH | Sense | CCCATCACCATCTTCCAGGAG |
| | Anti-sense | CTTCTCCATGGTGGTGAAGACG |
| GDNF | Sense | TCCATGACATCATCGAACTGA |
| | Anti-sense | GTCTGCCTGGTGCTGCTC |
| HoxA2 | Sense | CCAAGAAAACCGCACTTCTG |
| | Anti-sense | CATCGGCGATTTCCAGG |
| HoxB3 | Sense | CGTCATGAATGGGATCTGC |
| | Anti-sense | ATATTCACATCGAGCCCCAG |
| HoxB5 | Sense | GGAAGCTTCACATCAGCCAT |
| | Anti-sense | GGAACTCCTTTTCCAGCTCC |
| HoxB7 | Sense | AACTTCCGGATCTACCCCTG |
| | Anti-sense | CTTTCTCCAGCTCCAGGGTC |
| Pax3 | Sense | GCCGCATCCTGAGAAGTAAA |
| | Anti-sense | CTTCATCTGATTGGGGTGCT |
| Pax7 | Sense | CAAACACAGCATCGACGG |
| | Anti-sense | CTTCAGTGGGAGGTCAGGTT |
| Snai12 | Sense | TGACCTGTCTGCAAATGCTC |
| | Anti-sense | CAGACCCTGGTTGCTTCAA |
| Sox10 | Sense | AGCTCAGCAAGACGCTGG |
| | Anti-sense | CTTTCTTGTGCTGCATACGG |
| Sox9 | Sense | GTAATCCGGGTGGTCCTTCT |
| | Anti-sense | GTACCCGCACTTGCACAAC |
| Zic1 | Sense | AAGATCCACAAAAGGACGCA |
| | Anti-sense | CACGTGCATGTGCTTCTTG |

### Ex-Vivo Intestinal Motility

Engrafted HIO+/−NCCs were harvested and placed in ice-cold HBSS. Muscle strips (4-6 mm in length and 1-2 mm in width) were cut from the engrafted HIO+/−NCC. Preparations were suspended vertically in an organ bath filled with Krebs solution (NaCl, 117 mM; KCl, 4.7 mM; MgCl2, 1.2 mM; NaH2PO4, 1.2 mM; NaHCO3, 25 mM; CaCl2, 2.5 mM and glucose, 11 mM), warmed at 37° C. and gassed with 95% O2+5% CO2. After an equilibration period of 60 min at initial tension of 0.5 g, the contractile response of the muscle was continuously recorded, using 8-chamber tissue-organ bath with isometric force transducers (Radnoti) coupled to a computer equipped with LabChart software (AD Instruments). Muscle preparations were stimulated with dimethylphenylpiperazinium (DMPP; 10 μM; Sigma) and veratridine (3 μM; Sigma). Chemical stimulations were applied at 15 min intervals followed by 3 washes. Tetrodotoxin (TTX; 10 μM; Tocris) or NG-nitro-L-arginine methyl ester (L-NAME; 50 M; Sigma) was applied 5 min before DMPP stimulation. NOS was inhibited with sodium nitroprusside (SNP; 100 μM; Sigma). Methylene Blue (50 μM) was used to inhibit ICC activity. The effects of chemical stimulation on tension were evaluated by measuring the area under the curve (AUC). Data are expressed in AAUC i.e. “Stimulated” AUC measured 120 s after stimulation minus “Control” AUC measured 120 s before stimulation.

### Discussion

Applicant used principles of embryonic intestinal development to engineer human PSC-derived intestinal tissue containing a functional ENS. Human PSC-derived vagal-like NCCs that were recombined with PSC-derived intestinal organoids in 3 dimensional growth conditions migrated into intestinal mesenchyme, self-organized, and differentiated into an array of neuronal and glial cell types of the ENS. Following engraftment and growth in vivo, NCCs formed complex ganglionic structures and interganglionic fibers, similar to embryonic development of the myenteric and submucosal neural plexus. Applicant further demonstrated that the NCC-derived ENS was functionally integrated into intestinal smooth muscle and drove NO-dependent relaxation. The high degree of tissue organization seen in transplanted HIOs+NCCs suggests that the tissue that Applicant engineered in vitro had the intrinsic information for coordinated cell migration, proliferation, lineage commitment and assembly into a plexus that occurs during embryonic development of the ENS.

Vagal NCCs that give rise to the ENS derive from more posterior, HOX-positive regions of the neural tube. HOXB5, for example, is expressed in human NCCs during colonization of the gut and is required for formation of the ENS in mice. Studies in vertebrate embryos have demonstrated the importance of Wnt, FGF and retinoic acid (RA) signaling in neural patterning and formation of HOX-expressing NCCs. Applicant observed RA-induced expression of vagal HOX genes A2, B3, B5, and B7, whereas treatment with FGF4 had little effect (data not shown). Recent findings have observed that activation of Wnt signaling also promotes formation of NCCs capable of forming melanocytes, which is a hallmark of vagal/trunk NCCs 11, and Applicant also observed that activation of Wnt signaling can promote posterior NCC HOX gene expression, albeit to a lower extent than RA (data not shown). At the functional level, both PSC-derived cranial and vagal NCCs were capable of incorporating into HIOs and forming neuroglial lineages, which is consistent with studies done in chick-quail chimeric embryos where cranial NCCs could efficiently contribute to the ENS. Applicant observed differences in that cranial-like NCCs+HIOs also gave rise to pigmented cells and cartilage, consistent with the broader differentiation potential of cranial NCCs.

Applicant routinely observed the formation of a neuroglial plexus in transplanted HIOs+NCCs in close association with smooth muscle fibers with organization similar to the myenteric plexus. However, several lines of evidence suggest that the NCC-derived ENS was less mature and more fetal in nature. For example, development of a submucosal plexus appeared delayed in HIO+NCC tissues, and this may be similar to development in the human fetal gut, where the submucosal plexus develops 2-3 weeks after the myenteric plexus 26. Another indicator of developmental immaturity is that the neuroglial plexus in HIOs contained smaller nerve bundles than the adult human intestine, more similar to what is observed in the human fetal gut 16. The immature/fetal nature of HIO+NCC tissues may provide an opportunity to identify specific factors that regulate maturation of the fetal gut/ENS. For example it was recently demonstrated that the microbiota of the lumen influence the colonization of the mucosa by glial cells and the HIO+/−ENS model could allow for mechanistic dissection of this process. Factors that promote intestinal maturation could be used clinically with premature infants, who are at heightened risk for intestinal infections due to an immature mucosa.

Applicant observed a substantial degree of neuronal diversity in HIO+NCC tissue in vitro, including excitatory and interneurons that had intrinsic and inducible waves of calcium efflux, suggestive of neuronal activity. HIO+NCC tissues that were grown in vivo acquired additional neuronal diversity with nNOS+ inhibitory neurons, which are known to form at later stages in the developing fetal mouse gut. Moreover, neuroglial cells assembled into a myenteric and submucosal plexus that were functionally associated with highly differentiated layers of smooth muscle. Electrical field stimulation (EFS) of these engineered tissues triggered ENS-dependent waves of motility that were qualitatively similar to peristalsis. However, Applicant also observed intrinsic contractile activity that was dependent on resident ICCs that were present in both HIOs and HIO+NCC. Together our data suggest that ICCs were largely driving contraction whereas relaxation of muscle was ENS-dependent and mediated via nNOS+ inhibitory neurons present in HIO+NCC tissue. The fact that NOS inhibitory neurons became functional earlier than cholinergic excitatory neurons supports the conclusion the ENS in HIOs is fetal in nature.

There are several pronounced differences between rodents and humans regarding development, physiology and diseases of the intestine. For example, development of crypts occurs in utero in humans whereas it happens postnatally in mice. During ENS development there are differences in the formation of TH neurons between mouse and humans. Given that HIOs+NCCs contain TH positive neurons in vitro and in vivo, this system may be the only means to study the unique developmental properties of these cell types. Moreover, an experimental system to study human TH+ dopaminergic ENS neurons may provide insight into GI dysmotility symptoms found in Parkinson's patients 32 that is caused by degeneration of this neuronal subtype. Because of its highly tractable nature, this system should be particularly useful in studying developmental deficits of the ENS, for example Hirschprung's disease. Defects in NCC formation, migration, incorporation into the mesenchyme, and proliferation can all result in aganglionic sections of the intestine. Our ability to study the interactions between NCCs and intestinal organoid in vitro and to monitor and manipulate these interactions during formation of a functional ENS should allow for unprecedented mechanistic dissection of known and novel forms of Hirschprung's disease in humans.

### REFERENCES

Cheng, X. et al. Self-renewing endodermal progenitor lines generated from human pluripotent stem cells. Cell Stem Cell 10, 371-384 (2012).
Wells, J.M. & Spence, J.R. How to make an intestine. Development 141, 752-760 (2014).
Lahar, N. et al. Intestinal subepithelial myofibroblasts support in vitro and in vivo growth of human small intestinal epithelium. PLOS ONE 6, e26898 (2011).
Levin, D.E. et al. Human tissue-engineered small intestine forms from postnatal progenitor cells. J. Pediatr. Surg. 48, 129-137 (2013).
Spence, J.R. et al. Directed differentiation of human pluripotent stem cells into intestinal tissue in vitro. Nature 470, 105-109 (2011).
McCracken, K.W., Howell, J.C., Wells, J.M. & Spence, J.R. Generating human intestinal tissue from pluripotent stem cells in vitro. Nat. Protoc. 6, 1920-1928 (2011).
Williamson, R.C., Buchholtz, T.W. & Malt, R.A. Humoral stimulation of cell proliferation in small bowel after transection and resection in rats. Gastroenterology 75, 249-254 (1978).
Juno, R.J. et al. A serum factor after intestinal resection stimulates epidermal growth factor receptor signaling and proliferation in intestinal epithelial cells. Surgery 132, 377-383 (2002).
Juno, R.J., Knott, A.W., Erwin, C.R. & Warner, B.W. A serum factor(s) after small bowel resection induces intestinal epithelial cell proliferation: effects of timing, site, and extent of resection. J. Pediatr. Surg. 38, 868-874 (2003).
Simon-Assmann, P., Turck, N., Sidhoum-Jenny, M., Gradwohl, G. & Kedinger, M. In vitro models of intestinal epithelial cell differentiation. Cell Biol. Toxicol. 23, 241-256 (2007).
Jung, P. et al. Isolation and in vitro expansion of human colonic stem cells. Nat. Med. 17, 1225-1227 (2011).
Sato, T. et al. Long-term expansion of epithelial organoids from human colon, adenoma, adenocarcinoma, and Barrett's epithelium. Gastroenterology 141, 1762-1772 (2011).
Campbell, F.C., Tait, I.S., Flint, N. & Evans, G.S. Transplantation of cultured small bowel enterocytes. Gut 34, 1153-1155 (1993).
Agopian, V.G., Chen, D.C., Avansino, J.R. & Stelzner, M. Intestinal stem cell organoid transplantation generates neomucosa in dogs. J. Gastrointest. Surg. 13, 971-982 (2009).
Avansino, J.R., Chen, D.C., Hoagland, V.D., Woolman, J.D. & Stelzner, M. Orthotopic transplantation of intestinal mucosal organoids in rodents. Surgery 140, 423-434 (2006).
Tait, I.S., Evans, G.S., Flint, N. & Campbell, F.C. Colonic mucosal replacement by syngeneic small intestinal stem cell transplantation. Am. J. Surg. 167, 67-72 (1994).
Tait, I.S., Flint, N., Campbell, F.C. & Evans, G.S. Generation of neomucosa in vivo by transplantation of dissociated rat postnatal small intestinal epithelium. Differentiation 56, 91-100 (1994).
Fordham, R.P. et al. Transplantation of expanded fetal intestinal progenitors contributes to colon regeneration after injury. Cell Stem Cell 13,734-744 (2013).
Yui, S. et al. Functional engraftment of colon epithelium expanded in vitro from a single adult Lgr5(+) stem cell. Nat. Med. 18, 618-623 (2012).
Kosinski, C. et al. Indian hedgehog regulates intestinal stem cell fate through epithelial-mesenchymal interactions during development. Gastroenterology 139, 893-903 (2010).
McLin, V.A., Henning, S.J. & Jamrich, M. The role of the visceral mesoderm in the development of the gastrointestinal tract. Gastroenterology 136, 2074-2091 (2009).
Zorn, A.M. & Wells, J.M. Vertebrate endoderm development and organ formation. Annu. Rev. Cell Dev. Biol. 25, 221-251 (2009).
Gracz, A.D. et al. Brief report: CD24 and CD44 mark human intestinal epithelial cell populations with characteristics of active and facultative stem cells. Stem Cells 31, 2024-2030 (2013).
Kroon, E. et al. Pancreatic endoderm derived from human embryonic stem cells generates glucose-responsive insulin-secreting cells in vivo. Nat. Biotechnol. 26, 443-452 (2008).
Kovalenko, P.L. & Basson, M.D. The correlation between the expression of differentiation markers in rat small intestinal mucosa and the transcript levels of schlafen 3. JAMA Surg. 148, 1013-1019 (2013).
Dekaney, C.M. et al. Expansion of intestinal stem cells associated with long-term adaptation following ileocecal resection in mice. Am. J. Physiol. Gastrointest. Liver Physiol. 293, G1013-G1022 (2007).
D'Amour, K.A. et al. Efficient differentiation of human embryonic stem cells to definitive endoderm. Nat. Biotechnol. 23, 1534-1541 (2005).
Warlich, E. et al. Lentiviral vector design and imaging approaches to visualize the early stages of cellular reprogramming. Mol. Ther. 19, 782-789 (2011).
Wang, F. et al. Isolation and characterization of intestinal stem cells based on surface marker combinations and colony-formation assay. Gastroenterology 145, 383-395.e1-e21 (2013).
Groneberg, D.A. et al. Intestinal peptide transport: ex vivo uptake studies and localization of peptide carrier PEPT1. Am. J. Physiol. Gastrointest. Liver Physiol. 281, G697-G704 (2001).
Furness, J.B. The enteric nervous system and neurogastroenterology. Nature reviews. Gastroenterology & hepatology 9, 286-294 (2012).
Sasselli, V., Pachnis, V. & Burns, A.J. The enteric nervous system. Developmental biology 366, 64-73 (2012).
Obermayr, F., Hotta, R., Enomoto, H. & Young, H.M. Development and developmental disorders of the enteric nervous system. Nature reviews. Gastroenterology & hepatology 10, 43-57 (2013).
Saffrey, M.J. Cellular changes in the enteric nervous system during ageing. Developmental biology 382, 344-355 (2013).
Mckeown, S.J., Stamp, L., Hao, M.M. & Young, H.M. Hirschsprung disease: a developmental disorder of the enteric nervous system. Wiley interdisciplinary reviews. Developmental biology 2, 113-129 (2013).
Burns, A.J. & Thapar, N. Neural stem cell therapies for enteric nervous system disorders. Nature reviews. Gastroenterology & hepatology 11, 317-328 (2014).
Hao, M.M. & Young, H.M. Development of enteric neuron diversity. Journal of cellular and molecular medicine 13, 1193-1210 (2009).
Lancaster, M.A. & Knoblich, J.A. Organogenesis in a dish: modeling development and disease using organoid technologies. Science 345, 1247125 (2014).
Bajpai, R., et al. CHD7 cooperates with PBAF to control multipotent neural crest formation. Nature 463, 958-962 (2010).
Curchoe, C.L., et al. Early acquisition of neural crest competence during hESCs neuralization. PloS one 5, e13890 (2010).
Mica, Y., Lee, G., Chambers, S.M., Tomishima, M.J. & Studer, L. Modeling neural crest induction, melanocyte specification, and disease-related pigmentation defects in hESCs and patient-specific iPSCs. Cell reports 3, 1140-1152 (2013).
Kudoh, T., Wilson, S.W. & Dawid, I.B. Distinct roles for Fgf, Wnt and retinoic acid in posteriorizing the neural ectoderm. Development 129, 4335-4346 (2002).
Watson, C.L., et al. An in vivo model of human small intestine using pluripotent stem cells. Nature medicine 20, 1310-1314 (2014).
Fu, M., Tam, P.K., Sham, M.H. & Lui, V.C. Embryonic development of the ganglion plexuses and the concentric layer structure of human gut: a topographical study. Anatomy and embryology 208, 33-41 (2004).
Young, H.M., Ciampoli, D., Hsuan, J. & Canty, A.J. Expression of Ret-, p75(NTR)-, Phox2a-, Phox2b-, and tyrosine hydroxylase-immunoreactivity by undifferentiated neural crest-derived cells and different classes of enteric neurons in the embryonic mouse gut. Dev Dyn 216, 137-152 (1999).
Young, H.M., et al. GDNF is a chemoattractant for enteric neural cells. Developmental biology 229, 503-516 (2001).
Chen, T.W., et al. Ultrasensitive fluorescent proteins for imaging neuronal activity. Nature 499, 295-300 (2013).
Huebsch, N., et al. Automated Video-Based Analysis of Contractility and Calcium Flux in Human-Induced Pluripotent Stem Cell-Derived Cardiomyocytes Cultured over Different Spatial Scales. Tissue engineering. Part C, Methods 21, 467-479 (2015).
Fu, M., Lui, V.C., Sham, M.H., Cheung, A.N. & Tam, P.K. HOXB5 expression is spatially and temporarily regulated in human embryonic gut during neural crest cell colonization and differentiation of enteric neuroblasts. Dev Dyn 228, 1-10 (2003).
Lui, V.C., et al. Perturbation of hoxb5 signaling in vagal neural crests down-regulates ret leading to intestinal hypoganglionosis in mice. Gastroenterology 134, 1104-1115 (2008).
Denham, M., et al. Multipotent caudal neural progenitors derived from human pluripotent stem cells that give rise to lineages of the central and peripheral nervous system. Stem cells 33, 1759-1770 (2015).
Le Douarin, N.M., Creuzet, S., Couly, G. & Dupin, E. Neural crest cell plasticity and its limits. Development 131, 4637-4650 (2004).
Zhang, D., Brinas, I.M., Binder, B.J., Landman, K.A. & Newgreen, D.F. Neural crest regionalisation for enteric nervous system formation: implications for Hirschsprung's disease and stem cell therapy. Developmental biology 339, 280-294 (2010).
Wallace, A.S. & Burns, A.J. Development of the enteric nervous system, smooth muscle and interstitial cells of Cajal in the human gastrointestinal tract. Cell and tissue research 319, 367-382 (2005).
Kabouridis, P.S., et al. Microbiota controls the homeostasis of glial cells in the gut lamina propria. Neuron 85, 289-295 (2015).
Bergner, A.J., et al. Birthdating of myenteric neuron subtypes in the small intestine of the mouse. The Journal of comparative neurology 522, 514-527 (2014).
Baetge, G. & Gershon, M.D. Transient catecholaminergic (TC) cells in the vagus nerves and bowel of fetal mice: relationship to the development of enteric neurons. Developmental biology 132, 189-211 (1989).
Blaugrund, E., et al. Distinct subpopulations of enteric neuronal progenitors defined by time of development, sympathoadrenal lineage markers and Mash-1-dependence. Development 122, 309-320 (1996).
Anlauf, M., Schafer, M.K., Eiden, L. & Weihe, E. Chemical coding of the human gastrointestinal nervous system: cholinergic, VIPergic, and catecholaminergic phenotypes. The Journal of comparative neurology 459, 90-111 (2003).
Anderson, G., et al. Loss of enteric dopaminergic neurons and associated changes in colon motility in an MPTP mouse model of Parkinson's disease. Experimental neurology 207, 4-12 (2007).
Costa, M., et al. A method for genetic modification of human embryonic stem cells using electroporation. Nature protocols 2, 792-796 (2007).
Hockemeyer, D., et al. Genetic engineering of human pluripotent cells using TALE nucleases. Nature biotechnology 29, 731-734 (2011).
Tang, W., et al. Faithful expression of multiple proteins via 2A-peptide self-processing: a versatile and reliable method for manipulating brain circuits. The Journal of neuroscience: the official journal of the Society for Neuroscience 29, 8621-8629 (2009).
Lee, G., et al. Isolation and directed differentiation of neural crest stem cells derived from human embryonic stem cells. Nature biotechnology 25, 1468-1475 (2007).

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of making a vascularized hollow organ comprising the steps of
a) engrafting a human intestinal organoid (HIO) into an immune compromised mammal;
wherein said HIO is obtained from human embryonic stem cells (ESC's) and/or induced pluripotent stem cells (iPSCs);
wherein, during said engrafting step, said HIO forms mature intestinal tissue.

2. The method of claim 1, wherein said human intestinal organoid (HIO) is generated from human ESCs, or iPSCs, or a combination thereof.

3. The method of claim 1, wherein said engrafting step comprises transplantation of said HIO into the kidney capsule of said immune compromised organism.

4. The method of claim 1, wherein said engrafting step is carried out for a period of at least about three weeks, at least about four weeks, at least about five weeks, or at least about six weeks, or up to about three months, or up to about four months, or up to about five months, or up to about six months.

5. The method of claim 1, wherein said engraftment step is carried out until said intestinal tissue meets one or more criteria selected from having a columnar intestinal epithelium surrounded by a supporting mesenchyme, growth of 1-3 cm in diameter, the formation of villi and crypts containing functional intestinal cells, having submucosal and myenteric layers of smooth muscle fibers, and combinations thereof.

6. A method of making a human intestinal tissue containing a functional enteric nervous system (ENS) comprising the steps of
a) contacting vagal-like neural crest cells (NCCs) derived from human ES cells and/or iPS cells (IPCs) with a three dimensional human intestinal organoid (HIO); and
c) transplanting said HIO in vivo.

7. The method of claim 6, wherein said NCCs are obtained by contacting human ES cells and/or iPS cells with retinoic acid to cause posteriorization, optionally wherein said retinoic acid is contacted with said human ES cells and/or iPS cells for a period of about 1 to about 2 days, optionally about 2 days.

8. The method of claim 6, wherein said retinoic acid contacting step is carried out for a period of about two days at the neurosphere stage, or until substantial expression of HOXB3, HOXb5, and/or HOXb7 is observed.

9. The method of claim 6, wherein said transplanting step is carried out for a period of time sufficient to allow detection of neurons and/or glia.

10. The method of claim 6, wherein said neurons comprise BIII-tubulin.

11. The method of claim 6, wherein said glia comprise S100.

12. The method of claim 6, wherein said neurons and glia integrate into smooth muscle layers (desmin+ cells).

13. The method of claim 6, wherein said transplanting step is carried out for a period of time sufficient to allow formation of nNOS+ inhibitory neurons.

14. The method of claim 6, wherein said human intestinal tissue containing a functional enteric nervous system (ENS) is capable of contractile activity.

15. A method of treating a patient requiring replacement of a portion of a gastrointestinal tract of said patient, comprising the step of replacing said portion with a human intestinal tissue manufactured according to the method of claim 6.

16. A method of determining the effect of a treatment on a human intestinal tract, comprising the step of contacting said treatment with a human intestinal tissue according to the method of claim 6.
